SEQUENCE LISTING (65 sequences)

SEQ ID NO: 1 - DNA, 711 bp, Mus musculus
atggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctga

SEQ ID NO: 2 - protein, 236 aa, Mus musculus
MVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 3 - DNA, 1008 bp, Mus musculus
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctag

SEQ ID NO: 4 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 5 - DNA, 1444 bp, Homo sapiens
tttcattttgggccgagctggaggcggcggggccgtcccggaacggctgcggccgggcaccccgggagttaatccgaaagcgccgcaagccccgcgggccggccgcaccgcacgtgtcaccgagaagctgatgtagagagagacacagaaggagacagaaagcaagagaccagagtcccgggaaagtcctgccgcgcctcgggacaattataaaaatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccaaaccgttgtcatttttataaaactttgaaatgaggaaactttgataggatgtggattaagaactagggagggggaaagaaggatgggactattacatccacatgatacctctgatcaagtatttttgacatttactgtggataaattgtttttaagttttcatgaatgaattgctaagaagggaaaatatccatcctgaaggtgtttttcattcactttaatagaagggcaaatatttataagctatttctgtaccaaagtgtttgtggaaacaaacatgtaagcataacttattttaaaatatttatttatataacttggtaatcatgaaagcatctgagctaacttatatttatttatgttatatttattaaattatttatcaagtgtatttgaaaaatatttttaagtgttctaaaaataaaagtattgaattaaagtgaaaaaaaa

SEQ ID NO: 6 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 7 - DNA, 2347 bp, Homo sapiens
ctgtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaatgtgaatttaattttatttgtactgacaacttttcaagcaaggctgcaagtacatcagttttatgacaatcaggaagaatgcagtgttctgataccagtgccatcatacacttgtgatggatgggaacgcaagagatacttacatggaaacctgacaatgcaaacctgttgagaagatccaggagaacaagatgctagttcccatgtctgtgaagacttcctggagatggtgttgataaagcaatttagggccacttacacttctaagcaagtttaatctttggatgcctgaattttaaaagggctagaaaaaaatgattgaccagcctgggaaacataacaagaccccgtctctacaaaaaaaatttaaaattagccaggcgtggtggctcatgcttgtggtcccagctgttcaggaggatgaggcaggaggatctcttgagcccaggaggtcaaggctatggtgagccgtgattgtgccactgcataccagcctaggtgacagaatgagaccctgtctcaaaaaaaaaaatgattgaaattaaaattcagctttagcttccatggcagtcctcacccccacctctctaaaagacacaggaggatgacacagaaacaccgtaagtgtctggaaggcaaaaagatcttaagattcaagagagaggacaagtagttatggctaaggacatgaaattgtcagaatggcaggtggcttcttaacagccctgtgagaagcagacagatgcaaagaaaatctggaatccctttctcattagcatgaatgaacctgatacacaattatgaccagaaaatatggctccatgaaggtgctacttttaagtaatgtatgtgcgctctgtaaagtgattacatttgtttcctgtttgtttatttatttatttatttttgcattctgaggctgaactaataaaaactcttctttgtaatc

SEQ ID NO: 8 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 9 - DNA, 1176 bp, Artificial
  nucleic acid sequence encoding an IL-12p40-FG polypeptide
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccgggccggccggtggcggtgggagcggtggtgggggttccggaggcggagggtcggtgtcaaccatctcatcattctctccacctgggtaccaagtctctttctgcttggtgatggtactcctttttgcagtggacacaggactatatttctctgtgaagacaaacatttga

SEQ ID NO: 10 - protein, 391 aa, Artificial
  IL-12p40-FG polypeptide
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGPAGGGGSGGGGSGGGGSVSTISSFSPPGYQVSFCLVMVLLFAVDTGLYFSVKTNI

SEQ ID NO: 11 - DNA, 1203 bp, Artificial
  nucleic acid sequence encoding an IL-12p40-RG polypeptide
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccgggccggccgctgaagctgccgcaaaagaggccgctgcgaaggaggccgcggctaaggaggcggcagctaaagctgcagccgtgtcaaccatctcatcattctctccacctgggtaccaagtctctttctgcttggtgatggtactcctttttgcagtggacacaggactatatttctctgtgaagacaaacatttga

SEQ ID NO: 12 - protein, 400 aa, Artificial
  IL-12p40-RG polypeptide
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGPAAEAAAKEAAAKEAAAKEAAAKAAAVSTISSFSPPGYQVSFCLVMVLLFAVDTGLYFSVKTNI

SEQ ID NO: 13 - protein, 15 aa, Artificial
  peptide linker
GGGGSGGGGSGGGGS

SEQ ID NO: 14 - protein, 24 aa, Artificial
  peptide linker
AEAAAKEAAAKEAAAKEAAAKAAA

SEQ ID NO: 15 - protein, 6 aa, Artificial
  peptide linker
GGGGGG

SEQ ID NO: 16 - protein, 8 aa, Artificial
  peptide linker
GGGGGGGG

SEQ ID NO: 17 - protein, 5 aa, Artificial
  peptide linker
  REPEAT (1)..(5): residues 1-5 can be repeated 1-3 times
EAAAK

SEQ ID NO: 18 - protein, 46 aa, Artificial
  peptide linker
AEAAAKEAAAKEAAAKEAAAKALEAEAAAKEAAAKEAAAKEAAAKA

SEQ ID NO: 19 - protein, 5 aa, Artificial
  peptide linker
  REPEAT (1)..(5): residues 1-5 can be repeated 1, 2, or 4 times
GGGGS

SEQ ID NO: 20 - protein, 12 aa, Artificial
  peptide linker
AEAAAKEAAAKA

SEQ ID NO: 21 - protein, 5 aa, Artificial
  peptide linker
PAPAP

SEQ ID NO: 22 - protein, 20 aa, Artificial
  peptide linker
GGSGGSGGSGGSGGSGGSGG

SEQ ID NO: 23 - DNA, 40 bp, Artificial
  oligonucleotide primer
ggcggtcgacgagctcatgtgtcctcagaagctaaccatc

SEQ ID NO: 24 - DNA, 36 bp, Artificial
  oligonucleotide primer
ccgcgttaacctaggatcggaccctgcagggaacac

SEQ ID NO: 25 - DNA, 33 bp, Artificial
  oligonucleotide primer
ggcggtcgacatggtcagcgttccaacagcctc

SEQ ID NO: 26 - DNA, 34 bp, Artificial
  oligonucleotide primer
cgcgggcgcgcctcaggcggagctcagatagccc

SEQ ID NO: 27 - DNA, 40 bp, Artificial; identical to SEQ ID NO: 23
  oligonucleotide primer

SEQ ID NO: 28 - DNA, 36 bp, Artificial
  oligonucleotide primer
ccgcggccggcccggatcggaccctgcagggaacac

SEQ ID NO: 29 - DNA, 40 bp, Artificial; identical to SEQ ID NO: 23
  oligonucleotide primer

SEQ ID NO: 30 - DNA, 32 bp, Artificial
  oligonucleotide primer
gcgcgttaactcaaatgtttgtcttcacagag

SEQ ID NO: 31 - DNA, 33 bp, Artificial; identical to SEQ ID NO: 25
  oligonucleotide primer

SEQ ID NO: 32 - DNA, 34 bp, Artificial; identical to SEQ ID NO: 26
  oligonucleotide primer

SEQ ID NO: 33 - DNA, 60 bp, Artificial
  oligonucleotide primer
ggccggccggccgctgaagctgccgcaaaagaggccgctgcgaaggaggccgcggctaag

SEQ ID NO: 34 - DNA, 32 bp, Artificial; identical to SEQ ID NO: 30
  oligonucleotide primer

SEQ ID NO: 35 - DNA, 40 bp, Artificial; identical to SEQ ID NO: 23
  oligonucleotide primer

SEQ ID NO: 36 - DNA, 32 bp, Artificial; identical to SEQ ID NO: 30
  oligonucleotide primer

SEQ ID NO: 37 - DNA, 33 bp, Artificial; identical to SEQ ID NO: 25
  oligonucleotide primer

SEQ ID NO: 38 - DNA, 34 bp, Artificial; identical to SEQ ID NO: 26
  oligonucleotide primer

SEQ ID NO: 39 - protein, 20 aa, Artificial
  membrane anchoring polypeptide
SFSPPGYQVSFCLVMVLLFA

SEQ ID NO: 40 - protein, 34 aa, Artificial
  membrane anchoring polypeptide
SSFSPPGYQVSFCLVMVLLFAVDTGLYFSVKTNI

SEQ ID NO: 41 - protein, 47 aa, Artificial
  membrane anchoring polypeptide
CLEPYTACDLAPRAGTTDAAHPGPSVVPALLPLLAGTLLLLGTATAP

SEQ ID NO: 42 - protein, 48 aa, Artificial
  membrane anchoring polypeptide
TTCIPSSGHSRHRYALIPIPLAVITTCIVLYMNGILKCDRKPDRTNSN

SEQ ID NO: 43 - protein, 37 aa, Artificial
  membrane anchoring polypeptide
PNKGSGTTSGTTRLLSGHTCFTLTGLLGTLVTMGLLT

SEQ ID NO: 44 - protein, 32 aa, Artificial
  membrane anchoring polypeptide
YTVSNMATIAVVVDLGAVAIIGAVVAFVMNRR

SEQ ID NO: 45 - protein, 77 aa, Artificial
  membrane anchoring polypeptide
LQCYNCPNPTADCKTAVNCSSDFDACLITKAGLQVYNKCWKFEHCNFNDVTTRLRENELTYYCCKKDLCNFNEQLEN

SEQ ID NO: 46 - protein, 221 aa, Bos taurus
MCPLRSLLLISTLVLLHHLPHLSLGRSLPTTTASPGRSCLDYSQNLLRAVSNTLQKARQTLEFYSCTSEEIDHEDITKDKTSTVEACLPLELATNESCLASRETSFITNGHCLASGKTSFMTTLCLRSIYEDLKMYHVEFQAMNAKLLMDPKRQIFLDQNMLAAIAELMQALNFDSETVPQKPSLKELDFYKTKVKLCILLHAFRIRAVTIDRMMSYLSSS

SEQ ID NO: 47 - protein, 221 aa, Ovis aries
MCPLRSLLLISTLVLLHHLPHLSLGRSLPTTTAGPGTSCLDYSQNLLRAVSNTLQKARQTLEFYSCTSEEIDHEDITKDKTSTVEACLPLELATNESCLASRETSLITNGHCLSSGKTSFMTTLCLRSIYKDLKMYHMEFQAMNAKLLMDPKRQVFLDQNMLAAIAELMQALNFDSETVPQKPSLEELDFYKTKVKLCILLHAFRIRAVTIDRMMSYLSSS

SEQ ID NO: 48 - protein, 256 aa, Sus scrofa
MCSPGFGLQTSDLTCDVHPSASSRRLAVTAAPLSMCPLRNLLLVATLVLLNHLDHLSLGRSLPATTAGPGMFKCLNHSQNLLKAVSNTLQKAKQTLEFYSCTSEEIDHEDITKDKTSTVEACLPLELATNESCLAARETSLITNGNCLTSGKTSFMTTLCLSSIYEDLKMYHVEFQAMNAKLLMDPKRQIFLDQNMLTAITELMQALNFNSETVPQKPSLEELDFYKTKIKLCILLHAFRIRAVTIDRMMSYLNSS

SEQ ID NO: 49 - protein, 222 aa, Felis catus
MCPPRGLLLVTILVLLNHLDHLSLARNLPTPTPSPGMFQCLNHSQTLLRAISNTLQKARQTLEFYPCTSEEIDHEDITKDKTSTVEACLPLELTMNESCLASREISLITNGSCLASRKTSFMTTLCLSSIYEDLKMYQVEFKAMNAKLLMDPKRQIFLDQNMLTAIDELLQALNVNSVTVPQNSSLEEPDFYKTKIKLCILLHAFRIRAVTINRMMSYLNSS

SEQ ID NO: 50 - protein, 253 aa, Homo sapiens; identical to SEQ ID NO: 6

SEQ ID NO: 51 - protein, 253 aa, Papio anubis
MWPPGSASQPPPSPATATGLHPAARPVSLQCRLSMCPARSLLLVATLVLLDYLSLARNLSVATPGPEMFPCLHHSQNLLKAASNTLQKARQILEFYPCTSEEIDHEDITKDKTSTVEACLPLELIKNESCLNSRETSVITKGSCLASRKTSFMMALCLRSIYEDLQIYQVEFKTMNAKLLMDPKRQIFLDQNILGVIDELMQALNFNSETVPQKSSLEEPDFYKTKIRLCILLHAFRIRAVTIDRVMSYLNAS

SEQ ID NO: 52 - protein, 328 aa, Macaca mulatta
MCHQQLVISWFSLVFLASPLMAIWELKKDVYVVELDWYPDAPGEMVVLTCDTPEEDGITWTLDQSGEVLGSGKTLTIQVKEFGDAGQYTCHKGGEALSHSLLLLHKKEDGIWSTDVLKDQKEPKNKTFLRCEAKNYSGRFTCWWLTTISTDLTFSVKSSRGSSNPQGVTCGAVTLSAERVRGDNKEYEYSVECQEDSACPAAEERLPIEVMVDAIHKLKYENYTSSFFIRDIIKPDPPKNLQLKPLKNSRQVEVSWEYPDTWSTPHSYFSLTFCIQVQGKSKREKKDRIFTDKTSATVICRKNASFSVQAQDRYYSSSWSEWASVPCS

SEQ ID NO: 53 - protein, 236 aa, Mus musculus; identical to SEQ ID NO: 2

SEQ ID NO: 54 - protein, 215 aa, Rattus norvegicus
MCQSRYLLFLATLVLLNHLTSARVIPVSGPAKCLNQSQNLLKTTDDMVRTAREKLKHYSCTAGDIDHEDITRDKTSTLEACLPLELHKNESCLATKETSSIIRGSCLPPQKTSLMMTLCLGSIYEDLKMYQSEFQAINAALQSHNHQQITLDRNMLMAIDELMRSLNHSGETLHQKAPMGEADPYRVKMKLCILLHAFSTRVMTINRVMNYLSSS

SEQ ID NO: 55 - protein, 139 aa, Artificial
  consensus IL-12p35 polypeptide sequence
DITKDKTSTVEACLPLELNESCLASRETSITNGSCLASKTSFMMTLCLSIYEDLKMYQVEFQAMNAKLLMDPKRQIFLDQNMLAIDELMQALNFNSETVPQKSLEEDFYKTKIKLCILLHAFRIRAVTIDRMMSYLSSS

SEQ ID NO: 56 - protein, 250 aa, Bos taurus
MHPQQLVVSWFSLVLLASPIVAMWELEKNVYVVELDWYPDAPGETVVLTCDTPEEDGITWTSDQSSEVLGSGKTLTIQVKEFGDAGQYTCHKGGEALSRSLLLLHKKEDGIWSTDILKDQKEPKAKSFLKCEAKDYSGHFTCWWLTAISTDLKFSVKSSRGSSDPRGVTCGAALLSAEKVSLEHREYNKYTVECQEGSACPAAEESLLIEVVVEAVHKLKYENYTSSFFIRDIIKPDPPKNLQLRPLKNS

SEQ ID NO: 57 - protein, 327 aa, Ovis aries
MHPQQLVVSWFSLVLLASPIVAIWELEKNVYVVELDWYPNAPGETVVLTCDTPEEDGITWTSDQSSEVLGSGKTLTIQVKEFGDAGQYTCHKGGEVLSRSLLLLHKKEDGIWSTDILKDQKEPKAKSFLKCEAKDYSGHFTCSWLTAISTNLKFSVKSSRGSSDPRGVTCGAASLSAEKVSMDHREYNKYTVECQEGSACPAAEESLPIEVVMEAVHKLKYENYTSSFFIRDIIKPDPPKNLQLRPLKNSRQVEVSWEYPDTWSTPHSYFSLTFCVQVQGKNKREKKLFTDQTSAKVTCHKDANIRVQARDRYYSSFWSEWASVSCS

SEQ ID NO: 58 - protein, 324 aa, Sus scrofa
MHLQQLVVSWFSLVWLASPIVAIWELEKNVYVVELDWYPNAPGEMVVLTCNTPEEDGITWTSDQSSEVLGTGKTLTIHVKEFGDAGQYTCRKGGAVLSQSLLLLHKKEDGIWSTDILKDQKEPKNKSFLKCEAKNYSGRFTCWWLTAISTDLKFSVKSSRGSTDPRGVTCGTATLSEDLGEYKKYRVECQEGSACPAAEESLPIEVVLEAVHKLKYENYTSSFFIRDIIKPDPPKNLQLNPLKNSRHVEISWEYPDTWSTPHSYFSLMFGVQVQGKNKREKKDKLFTDQISAKVTCHKDANIRVQARDRYYSSSWSEWASVSCN

SEQ ID NO: 59 - protein, 329 aa, Felis catus
MHPQQLVIAWLSLVLLAPPLMAIWELEKNVYVVELDWHPDAPGEMVVLTCNTPEEDDITWTSDQSSEVLGSGKTLTIQVKEFADAGQYTCHKGGEVLSHSFLLIHKKEDGIWSTDILREQKESKNKIFLKCEAKNYSGRFTCWWLTAISTDLKFTVKSSRGSSDPQEVTCGAATLSAEKVRVDNRDYKKYTVECQEGSACPAAEESLPIEVVVDAIHKLKYENYTSSFFIRDIIKPDPPKNLQLKPLKNSRHVEVSWEYPDTWSTPHSYFSLTFGVQVQGKNNREKKDRLSVDKTSAKVVCHKDAKIRVQARDRYYSSSWSNWASVSCS

SEQ ID NO: 60 - protein, 328 aa, Homo sapiens; identical to SEQ ID NO: 8

SEQ ID NO: 61 - protein, 328 aa, Macaca mulatta; identical to SEQ ID NO: 52

SEQ ID NO: 62 - protein, 328 aa, Papio anubis
MCHQQLVISWFSLVFLASPLMAIWELKKDVYVVELDWYPDAPGEMVVLTCDTPEEDGITWTLDQSGEVLGSGKTLTIQVKEFGDAGQYTCHKGGEALSHSLLLLHKKEDGIWSTDVLKDQKEPKNKTFLRCEAKNYSGRFTCWWLTTISTDLTFSVKSSRGSSNPQGVTCGAVTLSAERVRGDNKEYEYSVECQEDSACPAAEERLPIEVMVDAIHKLKYENYTSSFFIRDIIKPDPPKNLQLKPLKNSRQAEVSWEYPDTWSTPHSYFSLTFCIQVQGKSKREKKDRIFTDKTSATVICRKNASFSVQAQDRYYSSSWSEWASVPCS

SEQ ID NO: 63 - protein, 335 aa, Mus musculus; identical to SEQ ID NO: 4

SEQ ID NO: 64 - protein, 335 aa, Rattus norvegicus
MCHQKLTFSWFAMVLLVSPLMAMWELEKDVYVVEVDWRPDAPGETVTLTCDSPEEDDITWTSDQRRGVIGSGKTLTITVREFLDAGQYTCHRGGETLSHSHLLLHKKENGIWSTEILKNFKNKTFLKCEAPNYSGRFTCSWLVHRNTDLKFNIKSSSSSPESRAVTCGRASLSAEKVTLNQRDYEKYSVACQEDVTCPTAEETLPIELVVEAQQQNKYENYSTSFFIRDIIKPDPPKNLQVKPLKNSQVEVSWEYPDSWSTPHSYFSLKFFVRIQRKKEKTKETEEECNQKGAFLVEKTSAEVQCKGANICVQAQDRYYNSSCSKWTCVPCRGRS

SEQ ID NO: 65 - protein, 319 aa, Artificial
  consensus IL-12p40 polypeptide sequence
MCQQLVISWFSLVLLASPLMAIWELEKDVYVVELDWYPDAPGEMVVLTCDTPEEDGITWTSDQSSEVLGSGKTLTIQVKEFGDAGQYTCHKGGELSHSLLLLHKKEDGIWSTDILKDQKEPKNKTFLKCEAKNYSGRFTCWWLTISTDLKFSVKSSRGSSDPRGVTCGAATLSAEKVLDNRDYKYSVECQEDSACPAAEESLPIEVVVEAVHKLKYENYTSSFFIRDIIKPDPPKNLQLKPLKNSRQVEVSWEYPDTWSTPHSYFSLTFCVQVQGKKREKKDRLFTDKTSAVCRKANIVQAQDRYYSSSWSEWASVPCS

(19) United States
(12) Patent Application Publication
(10) Pub. No.: US 2023/0002465 A1
(43) Pub. Date: Jan. 5, 2023

(54) VACCINIA VIRUSES AND METHODS FOR USING VACCINIA VIRUSES

(71) Applicant: University of Pittsburgh - Of the Commonwealth System of Higher Education, Pittsburgh, PA (US)

(72) Inventors: David Bartlett, Pittsburgh, PA (US); Zongsheng Guo, Wexford, PA (US); Zuqiang Liu, Wexford, PA (US)

(21) Appl. No.: 17/778,463

(22) PCT Filed: Nov. 20, 2020
(86) PCT No.: PCT/US2020/061578

§ 371 Date: May 20, 2022

Related U.S. Application Data
(60) Provisional application No. 62/938,094, filed on Nov. 20, 2019.

(51) Int. Cl. C07K 14/54; A61K 48/00; A61P 35/00; C12N 15/86

(52) U.S. Cl. CPC C07K 14/5434; A61K 48/0025; A61P 35/00; C12N 15/86; A61K 38/00

(57) ABSTRACT

The disclosure relates to methods and materials for treating cancer. For example, recombinant vaccinia viruses having the ability to direct the expression of membrane-bound IL-12 polypeptides on the surface of infected cells and methods for using such recombinant vaccinia viruses to treat cancer are provided. Specifically, the disclosure provides a recombinant vaccinia virus comprising a vaccinia virus genome comprising a nucleic acid encoding an IL-12p35 polypeptide sequence and an IL-12p40 polypeptide sequence, wherein one of the polypeptide sequences comprises a membrane anchoring polypeptide sequence.

### CLAIM OF PRIORITY

This application claims priority to U.S. Provisional Application No. 62/938,094, filed on Nov. 20, 2019, the entire contents of which is hereby incorporated by reference.

### BACKGROUND

### 1. Technical Field

This document relates to methods and materials for treating cancer. For example, this document provides recombinant vaccinia viruses and methods for using recombinant vaccinia viruses to treat medical conditions such as cancer. In some cases, recombinant vaccinia viruses provided herein can be used as an oncolytic agent (e.g., to treat cancer).

### 2. Background Information

Despite vast efforts, cancer remains a major public health issue in the United States with over 1.6 million new cases in 2017 alone (National Cancer Institute, “Cancer Stat Facts: Cancer of Any Site,” seer.cancer.gov/statfacts/html/all.html). Traditional therapies, such as chemotherapeutics, radiation therapy, and surgery, often fail, especially when a cancer is advanced. Cancer immunotherapy also can be used to treat cancer. However, while therapeutic responses can be observed after cancer immunotherapy, these successes are limited to a small percentage of patients (Hodi et al., N Engl. J. Med., 363:711-723 (2010); and Zou et al., Sci. Transl. Med., 8:328rv324 (2016)).

### SUMMARY

This document provides methods and materials for treating cancer. In some cases, this document provides recombinant vaccinia viruses having oncolytic anti-cancer activity. For example, recombinant vaccinia viruses having oncolytic anti-cancer activity can include nucleic acid encoding an interleukin 12 (IL-12) p35 polypeptide and nucleic acid encoding an IL-12p40 polypeptide where at least one of (or only one of) the IL-12p35 polypeptide and the IL-12p40 polypeptide includes a membrane anchoring polypeptide sequence. In some cases, one or more recombinant vaccinia viruses described herein (e.g., one or more recombinant vaccinia viruses having oncolytic anti-cancer activity) can be used as an oncolytic agent (e.g., to treat cancer). For example, one or more recombinant vaccinia viruses described herein can be administered to a mammal having cancer to treat that mammal.

Oncolytic virotherapy can provide an alternative approach to cancer treatment by utilizing recombinant vaccinia viruses to activate innate immunity (e.g., to induce immunogenic cell death) and/or to activate adaptive immunity (e.g., to provide life-long immunity against the tumors). As demonstrated herein, recombinant vaccinia viruses can be designed to include nucleic acid encoding a polycistronic transcript that can express an IL-12p35 polypeptide and an IL-12p40 polypeptide where at least one of (or only one of) the IL-12p35 polypeptide and the IL-12p40 polypeptide includes a membrane anchoring polypeptide sequence, and such recombinant vaccinia viruses can be used to infect cancer cells such that the cancer cell expresses the IL-12p35 polypeptide and the IL-12p40 polypeptide and such that the expressed IL-12p35 polypeptide and expressed IL-12p40 polypeptide form a membrane-bound IL-12 polypeptide (e.g., an IL-12p70 heterodimer including an IL-12p35 polypeptide and an IL-12p40 polypeptide). In some cases, recombinant vaccinia viruses including nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence can be used to convert non-T cell-inflamed tumors into T cell-inflamed tumors. For example, membrane-bound IL-12 polypeptides can facilitate tumor infiltration of more activated CD4⁺ and CD8⁺ T cells and less regulatory T cells (Tregs), granulocytic myeloid-derived suppressor cells (G-MDSCs), and exhausted CD8⁺ T cells, with increased expression of interferon-gamma (IFN-γ) and decreased expression of transforming growth factor beta (TGF-β), cyclooxygenase-2 (COX-2), and vascular endothelial growth factor (VEGF), leading to transformed, immunogenic tumors and improved survival. Also as demonstrated herein, recombinant vaccinia viruses including nucleic acid encoding a polycistronic transcript that can express an IL-12p35 polypeptide and an IL-12p40 polypeptide where at least one of (or only one of) the IL-12p35 polypeptide and the IL-12p40 polypeptide includes a membrane anchoring polypeptide sequence can be administered to a mammal without inducing systemic IL-12 toxicity. For example, vaccinia viruses engineered to include nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence can deliver that nucleic acid to tumor cells such that the tumor cells express one or more membrane-bound IL-12 polypeptides and such that the expressed membrane-bound IL-12 polypeptides are maintained attached to the tumor cells without inducing pulmonary edema. When recombinant vaccinia viruses including nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence are administered together with PD-1 blockade, effective antitumor responses can be observed in multiple tumor models. The results provided herein demonstrate that vaccinia viruses engineered to include nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence can be used as an oncolytic virotherapy for cancers.

Having the ability to convert “cold” tumors (e.g., non-T cell-inflamed tumors) into “hot” tumors (e.g., T cell-inflamed tumors) by administering one or more recombinant vaccinia viruses designed to include nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence as described herein can allow clinicians and patients to use vaccinia viruses as a safe and effective oncolytic virotherapy.

In general, one aspect of this document features a recombinant vaccinia virus comprising a vaccinia virus genome comprising (a) nucleic acid encoding a first polypeptide and (b) nucleic acid encoding a second polypeptide, wherein the first polypeptide comprises an IL-12p35 polypeptide sequence, wherein the second polypeptide comprises an IL-12p40 polypeptide, and wherein the first polypeptide or the second polypeptide comprises a membrane anchoring polypeptide sequence. The IL-12p35 polypeptide sequence can be a full length human IL-12p35 polypeptide sequence. The IL-12p35 polypeptide sequence can be a full length mouse IL-12p35 polypeptide sequence. The IL-12p40 polypeptide sequence can be a full length human IL-12p40 polypeptide sequence. The IL-12p40 polypeptide sequence can be a full length mouse IL-12p40 polypeptide sequence. The membrane anchoring polypeptide sequence can comprise a polypeptide having a glycosylphosphatidyl-inositol (GPI) modification. The membrane anchoring polypeptide sequence can be from about 10 amino acids to about 50 amino acids in length. The polypeptide having a GPI modification can be derived from a CD16b polypeptide. The CD16b polypeptide can be a human CD16b polypeptide. The first polypeptide can comprise the membrane anchoring polypeptide sequence. The first polypeptide can comprise a polypeptide linker between the IL-12p35 polypeptide sequence and the membrane anchoring polypeptide sequence. The second polypeptide can comprise the membrane anchoring polypeptide sequence. The second polypeptide can comprise a polypeptide linker between the IL-12p40 polypeptide sequence and the membrane anchoring polypeptide sequence. The polypeptide linker can be from about one amino acid to about 25 amino acids in length. The polypeptide linker can comprise a (G₄S)₃ sequence. The polypeptide linker can comprise an A(EA₃K)₄AAA (SEQ ID NO:14) sequence. The nucleic acid encoding the first polypeptide can be operable linked to a promoter capable of driving transcription of a polycistronic transcript that expresses the first polypeptide and the second polypeptide. The promoter can be selected from the group consisting of a p7.5 e/1 promoter and a pSe/1 promoter. The nucleic acid encoding the first polypeptide and the nucleic acid encoding the second polypeptide can be separated by an internal ribosome entry site (IRES). A cell expressing the first polypeptide and the second polypeptide can express the first polypeptide and the second polypeptide on its surface in the form of a heterodimer having the ability to stimulate an IL-12 receptor of another cell.

In another embodiment, this document features a method for treating a mammal having cancer. The method comprises (or consists essentially of or consists of) administering, to the mammal, a recombinant vaccinia virus, wherein the recombinant vaccinia virus is capable of infecting a cell and expressing a membrane-bound IL-12 polypeptide comprising a first polypeptide and a second polypeptide on a surface of the cell. The recombinant vaccinia virus can comprise a vaccinia virus genome comprising (a) nucleic acid encoding the first polypeptide and (b) nucleic acid encoding the second polypeptide, wherein the first polypeptide comprises an IL-12p35 polypeptide sequence, wherein the second polypeptide comprises an IL-12p40 polypeptide, and wherein the first polypeptide or the second polypeptide comprises a membrane anchoring polypeptide sequence. The IL-12p35 polypeptide sequence can be a full length human IL-12p35 polypeptide sequence. The IL-12p35 polypeptide sequence can be a full length mouse IL-12p35 polypeptide sequence. The IL-12p40 polypeptide sequence can be a full length human IL-12p40 polypeptide sequence. The IL-12p40 polypeptide sequence can be a full length mouse IL-12p40 polypeptide sequence. The membrane anchoring polypeptide sequence can comprise a polypeptide having a glycosylphosphatidyl-inositol (GPI) modification. The membrane anchoring polypeptide sequence can be from about 10 amino acids to about 50 amino acids in length. The polypeptide having a GPI modification can be derived from a CD16b polypeptide. The CD16b polypeptide can be a human CD16b polypeptide. The first polypeptide can comprise the membrane anchoring polypeptide sequence. The first polypeptide can comprise a polypeptide linker between the IL-12p35 polypeptide sequence and the membrane anchoring polypeptide sequence. The second polypeptide can comprise the membrane anchoring polypeptide sequence. The second polypeptide can comprise a polypeptide linker between the IL-12p40 polypeptide sequence and the membrane anchoring polypeptide sequence. The polypeptide linker can be from about one amino acid to about 25 amino acids in length. The polypeptide linker can comprise a (G₄S)₃ sequence. The polypeptide linker can comprise an A(EA₃K)₄AAA (SEQ ID NO:14) sequence. The nucleic acid encoding the first polypeptide can be operable linked to a promoter capable of driving transcription of a polycistronic transcript that expresses the first polypeptide and the second polypeptide. The promoter can be selected from the group consisting of a p7.5 e/1 promoter and a pSe/1 promoter. The nucleic acid encoding the first polypeptide and the nucleic acid encoding the second polypeptide can be separated by an internal ribosome entry site (IRES). A cell expressing the first polypeptide and the second polypeptide can express the first polypeptide and the second polypeptide on its surface in the form of a heterodimer having the ability to stimulate an IL-12 receptor of another cell. The mammal can be a human. The cell can be a cancer cell. The cell can be a stromal cell in a tumor microenvironment of the mammal. The cancer can be selected from the group consisting of colon cancer, lung cancer, prostate cancer, ovarian cancer, hepatocellular carcinoma, pancreatic cancer, kidney cancer, melanoma, brain cancer, lymphoma, myeloma, lymphocytic leukemia, myelogenous leukemia, and breast cancer. The administering step can comprise a systemic administration. The systemic administration can comprise an intraperitoneal administration. The method can further comprise administering, to the mammal, an immune checkpoint inhibitor. The immune checkpoint inhibitor can be selected from the group consisting of an anti-CTLA-4 antibody, an anti-CD28 antibody, an anti-PD-1 antibody, and an anti-PD-L1 antibody.

In another embodiment, this document features a method for increasing the number of activated T cells within a tumor microenvironment present in a mammal. The method comprises administering, to the mammal, a recombinant vaccinia virus, wherein a cell within the mammal expresses a membrane-bound IL-12 polypeptide comprising a first polypeptide and a second polypeptide on its surface, and wherein the number of activated T cells within the tumor microenvironment is increased. The recombinant vaccinia virus can comprise a vaccinia virus genome comprising (a) nucleic acid encoding the first polypeptide and (b) nucleic acid encoding the second polypeptide, wherein the first polypeptide comprises an IL-12p35 polypeptide sequence, wherein the second polypeptide comprises an IL-12p40 polypeptide, and wherein the first polypeptide or the second polypeptide comprises a membrane anchoring polypeptide sequence. The IL-12p35 polypeptide sequence can be a full length human IL-12p35 polypeptide sequence. The IL-12p35 polypeptide sequence can be a full length mouse IL-12p35 polypeptide sequence. The IL-12p40 polypeptide sequence can be a full length human IL-12p40 polypeptide sequence. The IL-12p40 polypeptide sequence can be a full length mouse IL-12p40 polypeptide sequence. The membrane anchoring polypeptide sequence can comprise a polypeptide having a glycosylphosphatidyl-inositol (GPI) modification. The membrane anchoring polypeptide sequence can be from about 10 amino acids to about 50 amino acids in length. The polypeptide having a GPI modification can be derived from a CD16b polypeptide. The CD16b polypeptide can be a human CD16b polypeptide. The first polypeptide can comprise the membrane anchoring polypeptide sequence. The first polypeptide can comprise a polypeptide linker between the IL-12p35 polypeptide sequence and the membrane anchoring polypeptide sequence. The second polypeptide can comprise the membrane anchoring polypeptide sequence. The second polypeptide can comprise a polypeptide linker between the IL-12p40 polypeptide sequence and the membrane anchoring polypeptide sequence. The polypeptide linker can be from about one amino acid to about 25 amino acids in length. The polypeptide linker can comprise a (G₄S)₃ sequence. The polypeptide linker can comprise an A(EA₃K)₄AAA (SEQ ID NO:14) sequence. The nucleic acid encoding the first polypeptide can be operable linked to a promoter capable of driving transcription of a polycistronic transcript that expresses the first polypeptide and the second polypeptide. The promoter can be selected from the group consisting of a p7.5 e/1 promoter and a pSe/1 promoter. The nucleic acid encoding the first polypeptide and the nucleic acid encoding the second polypeptide can be separated by an internal ribosome entry site (IRES). A cell expressing the first polypeptide and the second polypeptide can express the first polypeptide and the second polypeptide on its surface in the form of a heterodimer having the ability to stimulate an IL-12 receptor of another cell. The mammal can be a human. The activated T cell can be selected from the group consisting of CD4⁺ T cells, CD8⁺ T cells, and natural killer T cells.

In another embodiment, this document features a method for decreasing the number of suppressor T cells within a tumor microenvironment present in a mammal. The method comprises administering, to the mammal, a recombinant vaccinia virus, wherein a cell within the mammal expresses a membrane-bound IL-12 polypeptide comprising a first polypeptide and a second polypeptide on its surface, and wherein the number of suppressor T cells within the tumor microenvironment is decreased. The recombinant vaccinia virus can comprise a vaccinia virus genome comprising (a) nucleic acid encoding the first polypeptide and (b) nucleic acid encoding the second polypeptide, wherein the first polypeptide comprises an IL-12p35 polypeptide sequence, wherein the second polypeptide comprises an IL-12p40 polypeptide, and wherein the first polypeptide or the second polypeptide comprises a membrane anchoring polypeptide sequence. The IL-12p35 polypeptide sequence can be a full length human IL-12p35 polypeptide sequence. The IL-12p35 polypeptide sequence can be a full length mouse IL-12p35 polypeptide sequence. The IL-12p40 polypeptide sequence can be a full length human IL-12p40 polypeptide sequence. The IL-12p40 polypeptide sequence can be a full length mouse IL-12p40 polypeptide sequence. The membrane anchoring polypeptide sequence can comprise a polypeptide having a glycosylphosphatidyl-inositol (GPI) modification. The membrane anchoring polypeptide sequence can be from about 10 amino acids to about 50 amino acids in length. The polypeptide having a GPI modification can be derived from a CD16b polypeptide. The CD16b polypeptide can be a human CD16b polypeptide. The first polypeptide can comprise the membrane anchoring polypeptide sequence. The first polypeptide can comprise a polypeptide linker between the IL-12p35 polypeptide sequence and the membrane anchoring polypeptide sequence. The second polypeptide can comprise the membrane anchoring polypeptide sequence. The second polypeptide can comprise a polypeptide linker between the IL-12p40 polypeptide sequence and the membrane anchoring polypeptide sequence. The polypeptide linker can be from about one amino acid to about 25 amino acids in length. The polypeptide linker can comprise a (G₄S)₃ sequence. The polypeptide linker can comprise an A(EA₃K)₄AAA (SEQ ID NO:14) sequence. The nucleic acid encoding the first polypeptide can be operable linked to a promoter capable of driving transcription of a polycistronic transcript that expresses the first polypeptide and the second polypeptide. The promoter can be selected from the group consisting of a p7.5 e/1 promoter and a pSe/1 promoter. The nucleic acid encoding the first polypeptide and the nucleic acid encoding the second polypeptide can be separated by an internal ribosome entry site (IRES). A cell expressing the first polypeptide and the second polypeptide can express the first polypeptide and the second polypeptide on its surface in the form of a heterodimer having the ability to stimulate an IL-12 receptor of another cell. The mammal can be a human. The suppressor T cell is selected from the group consisting of regulatory T cells (Tregs), granulocytic myeloid-derived suppressor cells (G-MDSCs), and exhausted CD8⁺ T cells.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. (A) An exemplary nucleic acid sequence (SEQ ID NO:1) encoding a murine IL-12p35 polypeptide and an exemplary amino acid sequence of a murine IL-12p35 polypeptide (SEQ ID NO:2). (B) An exemplary nucleic acid sequence (SEQ ID NO:3) encoding a murine IL-12p40 polypeptide and an exemplary amino acid sequence of a murine IL-12p40 polypeptide (SEQ ID NO:4). (C) An exemplary nucleic acid sequence (SEQ ID NO:5) encoding a human IL-12p35 polypeptide and an exemplary amino acid sequence of a human IL-12p35 polypeptide (SEQ ID NO:6). (D) An exemplary nucleic acid sequence (SEQ ID NO:7) encoding a human IL-12p40 polypeptide and an exemplary amino acid sequence of a human IL-12p40 polypeptide (SEQ ID NO:8). (E) A nucleic acid sequence (SEQ ID NO:9) encoding an IL-12p40-FG polypeptide and an amino acid sequence of an IL-12p40-FG polypeptide (SEQ ID NO:10). Single-underlining identifies sequences for a flexible linker, and double-underlining identifies sequences for a GPI anchor sequence. (F) A nucleic acid sequence (SEQ ID NO:11) encoding an IL-12p40-RG polypeptide and an amino acid sequence of an IL-12p40-RG polypeptide (SEQ ID NO:12). Single-underlining identifies sequences for a rigid linker, and double-underlining identifies sequences for a GPI anchor sequence. (G) A schematic diagram of viral IL-12 variants designed for insertion into a thymidine kinase (tk) locus using a right tk locus homology arm (TKR) and a left tk locus homology arm (TKL). vvDD-IL-12, vvDD-IL-12-FG, and vvDD-IL-12-RG recombinant vaccinia viruses were generated by homologous recombination of murine IL-12 variants into the tk locus of vaccinia viral genome, carrying secreted IL-12, IL-12-flexible linker (G₄S)₃-GPI anchor sequence (SEQ ID NO:9) amplified from human CD16b, and IL-12-rigid linker A(EA₃K)₄AAA-GPI anchor sequence (SEQ ID NO:11) amplified from human CD16b, respectively.

FIG. 2. Tethered IL-12 variants show functional IL-12 membrane association and similar cytotoxicity. (A) Tumor cells of MC38-luc (3×10⁵ cells), B16 (2×10⁵ cells), or AB12-luc (3×10⁵ cells) were mock-infected or infected with vvDD, vvDD-IL-12, vvDD-IL-12-FG, or vvDD-IL-12-RG at an MOI of 1. The cell pellets were harvested to measure A34R or IL-12 expression at 24 hours using RT-qPCR. (B, C) MC38-luc (3×10⁵ cells), B16 (2×10⁵ cells), or AB12-luc (3×10⁵ cells) were mock-infected or infected with vvDD, vvDD-IL-12, vvDD-IL-12-FG, or vvDD-IL-12-RG at an MOI of 1. The culture supernatants were harvested to measure secreted IL-12 using ELISA (B) and the cell pellets were harvested to measure membrane-bound IL-12 using flow cytometry (cell surface staining) (C) 24 hours post-infection. (D) MC38-luc (3×10⁵ cells), B16 (2×10⁵ cells), or AB12-luc (3×10⁵ cells) were mock-infected or infected with vvDD, vvDD-IL-12, vvDD-IL-12-FG, or vvDD-IL-12-RG at MOIs of 0.1, 1, and 5. The cell pellets were harvested to measure membrane-bound IL-12 using ELISA after PI-PLC cleavage 24 hours post-infection. (E) Naïve B6 splenocytes were activated and stimulated with IL-12 variant-infected MC38 cells (responder:stimulator=1:5) in the absence/presence of α-IL-12 Ab, and T cell proliferation was measured using MTT. (F) MC38-luc (1×10⁴ cells), B16 (5×10³ cells), AB12-luc (5×10³ cells), or CT26-luc (1×10⁴ cells) were infected with IL-12-variants at indicated MOIs and cell viability was measured using MTS assay. Data represent two independent experiments. * P<0.05; ** P<0.01; *** P<0.001; and **** P<0.0001. ns: not significant.

FIG. 3. Viral delivered IL-12 expression in tumor cells. Tumor cell MC38-luc (3×10⁵ cells) (top panel), AB12-luc (3×10⁵ cells) (middle panel), or B16 (2×10⁵ cells) (bottom panel) were mock-infected or infected with vvDD, vvDD-IL-12, vvDD-IL-12-FG, and vvDD-IL-12-RG at a MOI of 1. The cell pellets were harvested to measure membrane-bound IL-12 using flow cytometry (cell surface staining).

FIG. 4. vvDD-IL-12-FG treatment produces tethered IL-12 in tumors and is safe and effective in therapeutic tumor models. (A and B) B6 mice were intraperitoneally (i.p.) inoculated with 5×10⁵ MC38-luc cells and treated with PBS, vvDD, vvDD-IL-12, vvDD-IL-12-FG, or vvDD-IL-12-RG at 5×10⁸PFU/mouse nine days post-tumor inoculation (four mice/group). Sera were collected daily until day 5 to measure the amount of IL-12 (A) and IFN-γ (B) in sera. (C and D) The mice treated above were sacrificed at day 5 to measure IL-12 membrane association in tumor using flow cytometry and to monitor pulmonary edema. (E) B6 mice were i.p. inoculated with 5×10⁵ MC38-luc cells and treated with PBS, vvDD, vvDD-IL-12, or vvDD-IL-12-FG at 2×10⁸PFU/mouse nine days post-tumor inoculation (>13 mice/group, pooled). The mice treated above were sacrificed at day 5 to monitor pulmonary edema. (F) B6 mice were i.p inoculated with 5×10⁵ MC38-luc cells and treated with PBS, vvDD, vvDD-IL-12, or vvDD-IL-12-FG at 2×10⁸PFU/mouse five days post-tumor inoculation (n=8 or more). (G, H) The vvDD-IL-12-FG cured mice were subcutaneously re-challenged with MC38 or LLC. (I) B6 mice were i.p inoculated with 5×10⁵ MC38-luc cells and treated with PBS or indicated viruses at 2×10⁸PFU/mouse nine days post-tumor inoculation (n=23 or more). (J) BalB/c mice were i.p inoculated with 4×10⁵ AB12-luc cells and treated with PBS or indicated viruses at 2×10⁸PFU/mouse nine days post-tumor inoculation (n=10 or more). A log-rank (Mantel-Cox) test was used to compare survival rates. * P<0.05; ** P<0.01; *** P<0.001; and **** P<0.0001. ns: not significant.

FIG. 5. IL-12 variants elicit antitumoral effects in mouse colon and mesothelioma models. BalB/c mice were i.p. inoculated with 4×10⁵ CT26-luc (A) or AB12-luc cells (B), respectively, and treated with PBS, vvDD, vvDD-IL-12, or vvDD-IL-12-FG at 2×10⁸ PFU/mouse five days post-tumor inoculation and a log-rank (Mantel-Cox) test was used to compare survival rates between these two tumor models.

FIG. 6. IL-12-variant treatments change immune profile in the tumor microenvironment. B6 mice were inoculated i.p. with 5×10⁵ MC38-luc cells and treated with PBS, vvDD, vvDD-IL-12, or vvDD-IL-12-FG at 2×10⁸PFU/mouse nine days post-tumor inoculation. Tumor-bearing mice were sacrificed five days post-treatment and primary tumors were collected and analyzed using flow cytometry to determine CD4⁺Foxp3⁻ (A) and CD8⁺ T cells (B), exhausted CD8⁺ T cell (C-E), G-MDSCs (F), CD8/G-MDSCs (G), or regulatory T cells (CD4⁺Foxp3⁺) (H) using RT-qPCR to determine IFN-γ, granzyme B, PD-1, PD-L1, TGF-β, COX-2, CD105, and VEGF (I-O). In a separate experiment, B6 mice were i.p. inoculated with 5×10⁵ MC38-luc cells and treated with vvDD-IL-12-FG or PBS nine days post-tumor inoculation. α-CD8 Ab (250 μg per injection), α-CD4 Ab (150 μg per injection), α-IFN-γ Ab (200 μg per injection), or PK136 (300 μg per injection) (n=7 or) were i.p. injected into mice to deplete CD8⁺ T cells, CD4⁺ T cells, or NK1.1+ cells, or neutralize circulating IFN-γ (P), and a log-rank (Mantel-Cox) test was used to compare survival rates (Q), respectively. * P<0.05; ** P<0.01; *** P<0.001; and **** P<0.0001. ns: not significant.

FIG. 7. IL-12-variants produce IL-12 in tumor. B6 mice were i.p. inoculated with 5×10⁵ MC38-luc cells and treated with PBS, vvDD, vvDD-IL-12, or vvDD-IL-12-FG at 2×10⁸PFU/mouse nine days post-tumor inoculation. Primary tumors were harvested five days post treatment and applied to extract RNA for RT-qPCR to determine the expression of IL-12p40. * P<0.05; ** P<0.01; ***P<0.001; and **** P<0.0001. ns: not significant.

FIG. 8. Tethered IL-12 variant synergistically works with PD-1 blockade to potentiate antitumor effects. B6 mice were inoculated with 5×10⁵ MC38-luc cells and treated with indicated viruses at 2×10⁸ PFU/mouse nine days post-tumor inoculation alone or with α-PD-1 Ab (200 g/injection) as scheduled (A) and a log-rank (Mantel-Cox) test was used to compare survival rates in three tumor models (B-D). * P<0.05; ** P<0.01; *** P<0.001; and **** P<0.0001. ns: not significant.

FIG. 9. Amino acid sequence alignment of a representative group of full length IL-12p35 polypeptides (SEQ ID NOs:46-54 from top to bottom) and a consensus IL-12p35 polypeptide (SEQ ID NO:55).

FIG. 10. Amino acid sequence alignment of a representative group of full length IL-12p40 polypeptides (SEQ ID NOs:56-64 from top to bottom) and a consensus IL-12p40 polypeptide (SEQ ID NO:65).

### DETAILED DESCRIPTION

This document provides methods and materials for treating cancer. For example, this document provides methods and materials for treating cancer using one or more recombinant vaccinia viruses as an oncolytic agent. In some cases, this document provides recombinant vaccinia viruses having oncolytic anti-cancer activity. For example, a recombinant vaccinia virus having oncolytic anti-cancer activity can include (e.g., can be designed to include) nucleic acid encoding an IL-12p36 polypeptide and nucleic acid encoding an IL-12p40 polypeptide where at least one of (or only one of) the IL-12p35 polypeptide and the IL-12p40 polypeptide includes a membrane anchoring polypeptide sequence. In some cases, this document provides methods for using one or more recombinant vaccinia viruses described herein to treat a mammal having, or at risk of having, cancer. For example, one or more recombinant vaccinia viruses described herein can be administered to a mammal having, or at risk of having, cancer to reduce the number of cancer cells (e.g., by infecting and killing cancer cells) in the mammal (e.g., a human). For example, one or more recombinant vaccinia viruses described herein can be administered to a mammal having, or at risk of having, cancer to stimulate anti-cancer immune responses in the mammal (e.g., a human).

In some cases, a recombinant vaccinia virus described herein (e.g., recombinant vaccinia viruses having oncolytic anti-cancer activity) can be replication competent. For example, a recombinant vaccinia virus provided herein can replicate in (e.g., infect and kill) a cancer cell. In some cases, a recombinant vaccinia virus provided herein can replicate in (e.g., infect and kill) a stromal cell (e.g., a stromal cell present in a tumor microenvironment (TME)).

In some cases, a recombinant vaccinia virus described herein (e.g., recombinant vaccinia viruses having oncolytic anti-cancer activity) can be replication defective (e.g., can be replication defective in non-cancerous cells).

In some cases, a recombinant vaccinia virus described herein (e.g., recombinant vaccinia viruses having oncolytic anti-cancer activity) can be non-pathogenic (e.g., non-pathogenic to a mammal being treated as described herein).

In some cases, a recombinant vaccinia virus described herein (e.g., recombinant vaccinia viruses having oncolytic anti-cancer activity) can infect dividing cells (e.g., can infect only dividing cells). For example, a recombinant vaccinia virus can infect a dividing cancer cell.

A recombinant vaccinia virus described herein (e.g., a recombinant vaccinia virus having oncolytic anti-cancer activity) can be derived from any appropriate vaccinia virus. Examples of vaccinia viruses from which a recombinant vaccinia virus described herein can be derived from include, without limitation, a Western Reserve strain of vaccinia virus, a Wyeth strain of vaccinia virus, a Lederle-Chorioallantoic strain of vaccinia virus, a CL strain of vaccinia virus, a Lister strain of vaccinia virus, a MVA strain of vaccinia virus, a Dryvax strain of vaccinia virus, a Copenhagen strain of vaccinia virus, and a Tian Tan strain of vaccinia virus. In some cases, a recombinant vaccinia virus can be derived from a Western Reserve strain of vaccinia virus.

A recombinant vaccinia virus described herein can be any appropriate recombinant vaccinia virus (e.g., a recombinant vaccinia virus having oncolytic anti-cancer activity). In some cases, a recombinant vaccinia virus can be any vaccinia virus generated by recombining material (e.g., nucleic acids and/or polypeptides) from any organism other than the vaccinia virus from which the recombinant vaccinia virus is derived. For example, a recombinant vaccinia virus can include one or more materials that do not naturally occur in that vaccinia virus (e.g., do no naturally occur in that vaccinia virus prior to recombination). In some cases, a recombinant vaccinia virus provided herein can be a chimeric vaccinia virus (e.g., can include viral elements from two or more (e.g., two, three, four, five, or more) different vaccinia virus genomes). Nucleic acids that do not naturally occur in the vaccinia virus can be from any appropriate source. In some cases, nucleic acid that does not naturally occur in that vaccinia virus can be from a non-viral organism. In some cases, a nucleic acid that does not naturally occur in that vaccinia virus can be from a virus other than a vaccinia virus. In some cases, nucleic acid that does not naturally occur in that vaccinia virus can be from a different strain of vaccinia virus (e.g., a serotypically distinct strain). In some cases, nucleic acid that does not naturally occur in that vaccinia virus can be synthetic nucleic acid.

In some cases, a recombinant vaccinia virus described herein (e.g., recombinant vaccinia viruses having oncolytic anti-cancer activity) can include nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence. For example, a recombinant vaccinia virus described herein can include nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence such that, when the recombinant vaccinia virus infects a cell, the infected cell expresses the IL-12p35 polypeptide and the IL-12p40 polypeptide, the IL-12p35 polypeptide and the IL-12p40 polypeptide complex to form a membrane-bound IL-12 polypeptide (e.g., a membrane-bound IL-12p70 heterodimer including an IL-12p35 polypeptide and an IL-12p40 polypeptide), and the membrane-bound IL-12 polypeptide is presented on the surface (e.g., the membrane) of the infected cell. In some cases, a single nucleic acid sequence can encode a polycistronic transcript that can express both an IL-12p35 polypeptide and an IL-12p40 polypeptide where at least one of (or only one of) the IL-12p35 polypeptide and the IL-12p40 polypeptide includes a membrane anchoring polypeptide sequence. In some cases, nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence can be separate nucleic acid sequences.

A membrane-bound IL-12 polypeptide can include an IL-12p35 polypeptide and an IL-12p40 polypeptide, where at least one of (or only one of) the IL-12p35 polypeptide and the IL-12p40 polypeptide includes a membrane anchoring polypeptide sequence. For example, a membrane-bound IL-12 polypeptide can be a multimeric polypeptide that includes (a) an IL-12p35 polypeptide and (b) an IL-12p40 polypeptide fused to a membrane anchoring polypeptide sequence. In some cases, a membrane-bound IL-12 polypeptide can be a multimeric polypeptide that includes (a) an IL-12p35 polypeptide fused to a membrane anchoring polypeptide sequence and (b) an IL-12p40 polypeptide.

A recombinant vaccinia virus described herein (e.g., a recombinant vaccinia virus having oncolytic anti-cancer activity) can include nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence inserted into any appropriate location within the vaccinia virus genome. Examples of locations in a vaccinia virus genome into which nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence can be inserted include, without limitation, a tk locus (e.g., nucleic acid encoding a TK polypeptide), a vaccinia growth factor (vgf) locus (e.g., nucleic acid encoding a VGF polypeptide), a K3L locus (e.g., nucleic acid encoding a K3L polypeptide), a A56R locus (e.g., nucleic acid encoding a A56R polypeptide), a B18R locus (e.g., nucleic acid encoding a B18R polypeptide), and a M2L locus (e.g., nucleic acid encoding a M2L polypeptide). In some cases, nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence can be inserted into a tk locus of a genome of a recombinant vaccinia virus described herein. As described herein, when nucleic acid encoding an IL-12p35 polypeptide and nucleic acid encoding an IL-12p40 polypeptide including a membrane anchoring polypeptide sequence is inserted into a tk locus of a genome of a recombinant vaccinia virus described herein and the recombinant vaccinia virus is used to infect a cell, the nucleic acid encoding the IL-12p35 polypeptide can express the IL-12p35 polypeptide and the nucleic acid encoding the IL-12p40 polypeptide including a membrane anchoring polypeptide sequence can express the IL-12p40 polypeptide including a membrane anchoring polypeptide sequence such that the IL-12p35 polypeptide and the IL-12p40 polypeptide including a membrane anchoring polypeptide sequence can form a membrane-bound IL-12 polypeptide, and the membrane-bound IL-12 polypeptide can be presented on the surface (e.g., the membrane) of the infected cell.

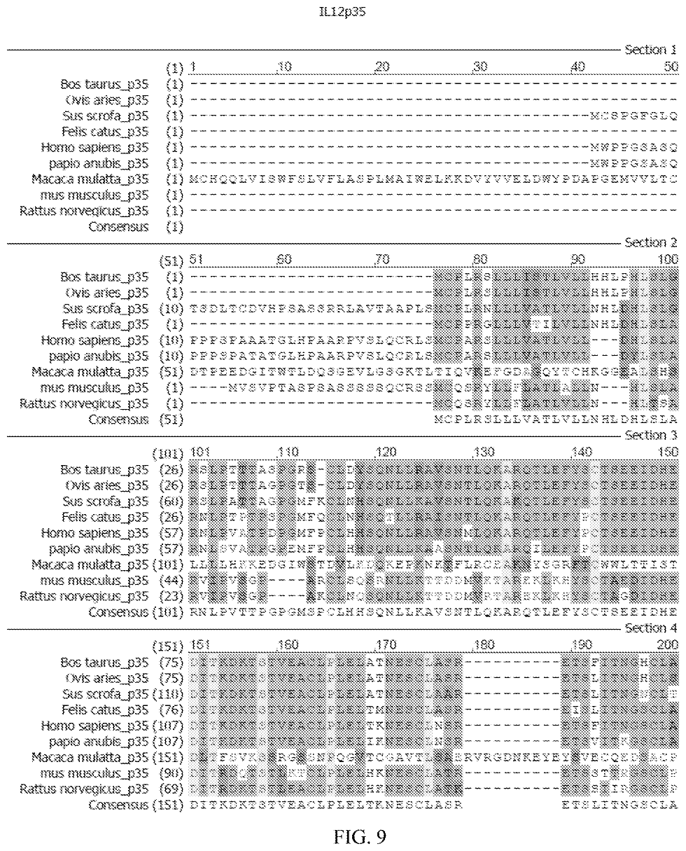

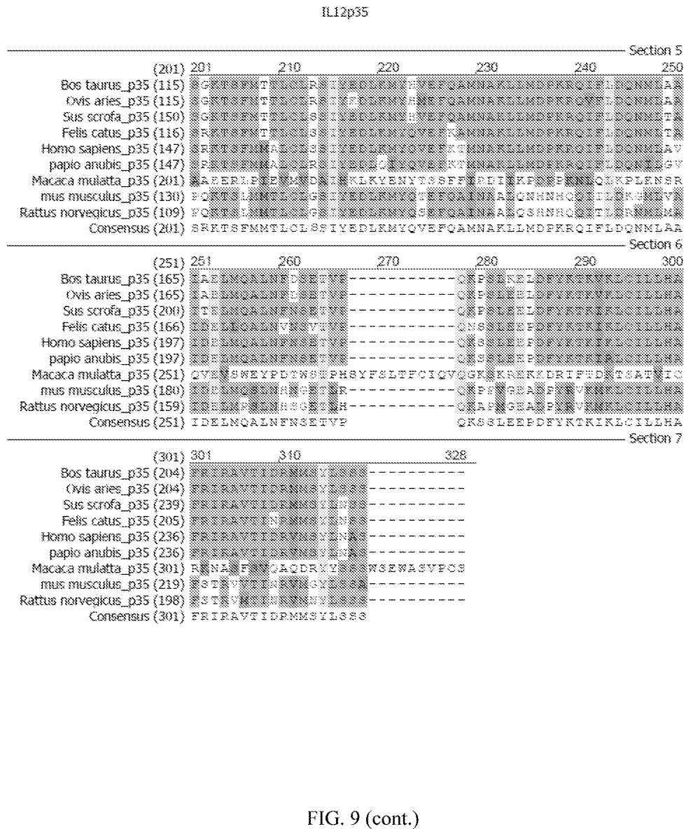

Nucleic acid encoding an IL-12p35 polypeptide that can be incorporated into a vaccinia virus as described herein can be designed to encode any appropriate IL-12p35 polypeptide. For example, full length IL-12p35 polypeptide sequences can be used as a part of a membrane-bound IL-12 polypeptide. Examples of full length IL-12p35 polypeptide sequences that can be used as a part of a membrane-bound IL-12 polypeptide described herein include, without limitation, those set forth in FIG. 9. In some cases, a consensus polypeptide comprising or consisting of the amino acid sequence of SEQ ID NO:55 can be used as described herein in place of a full length IL-12p35 polypeptide sequence. In some cases, a biologically active fragment of a full length IL-12p35 polypeptide sequence can be used as described herein in place of a full length IL-12p35 polypeptide sequence.

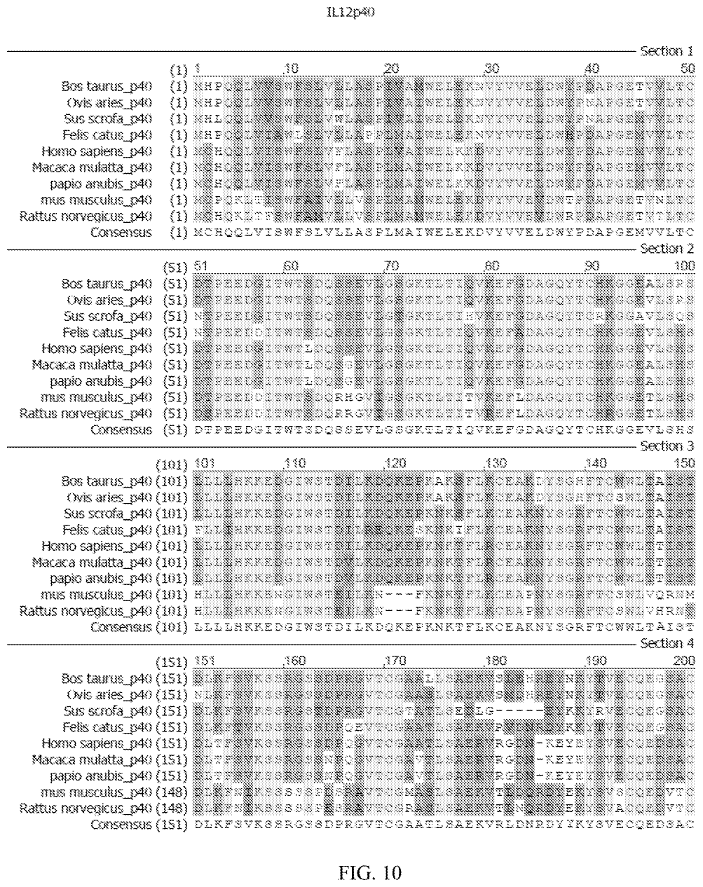

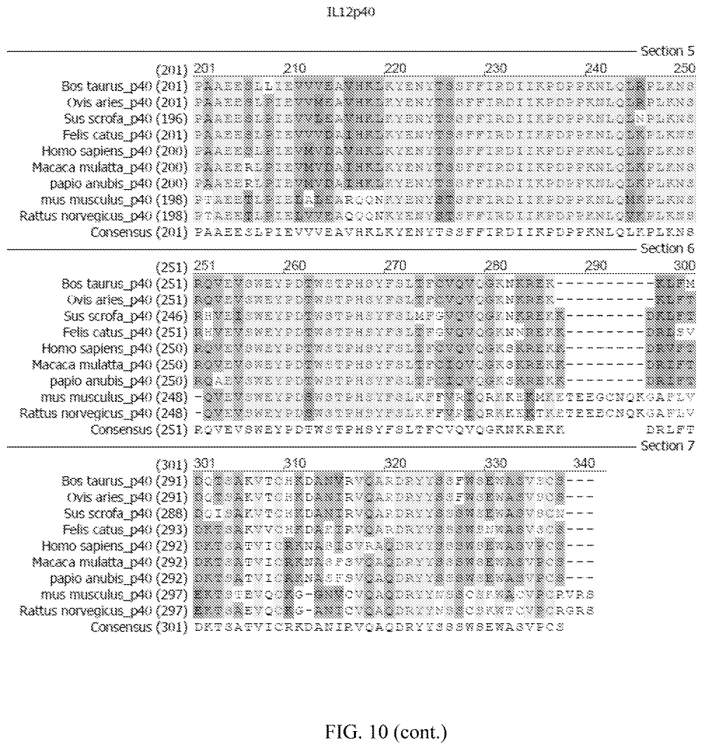

Nucleic acid encoding an IL-12p40 polypeptide that can be incorporated into a vaccinia virus as described herein can be designed to encode any appropriate IL-12p40 polypeptide. For example, full length IL-12p40 polypeptide sequences can be used as a part of a membrane-bound IL-12 polypeptide. Examples of full length IL-12p40 polypeptide sequences that can be used as a part of a membrane-bound IL-12 polypeptide described herein include, without limitation, those set forth in FIG. 10. In some cases, a consensus polypeptide comprising or consisting of the amino acid sequence of SEQ ID NO:65 can be used as described herein in place of a full length IL-12p40 polypeptide sequence. In some cases, a biologically active fragment of a full length IL-12p40 polypeptide sequence can be used as described herein in place of a full length IL-12p40 polypeptide sequence.

An IL-12p35 polypeptide sequence and an IL-12p40 polypeptide sequence of a membrane-bound IL-12 polypeptide can be from any appropriate species. In some cases, an IL-12p35 polypeptide sequence and an IL-12p40 polypeptide sequence of a membrane-bound IL-12 polypeptide can be mammalian sequences. In some cases, an IL-12p35 polypeptide sequence and an IL-12p40 polypeptide sequence of a membrane-bound IL-12 polypeptide can be from the same species. In some cases, an IL-12p35 polypeptide sequence and an IL-12p40 polypeptide sequence of a membrane-bound IL-12 polypeptide can be different species. Examples of species from which an IL-12p35 polypeptide sequence and an IL-12p40 polypeptide sequence can be obtained include, without limitation, humans and mice. In some cases, an IL-12p35 polypeptide sequence and an IL-12p40 polypeptide sequence of a membrane-bound IL-12 polypeptide can be from the same species as a mammal to be treated by administering one or more recombinant vaccinia viruses including nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence. For example, when a mammal to be treated by administering one or more recombinant vaccinia viruses including nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence is a human, the nucleic acid encoding an IL-12p35 polypeptide can encode a full length human IL-12p35 polypeptide (or a biologically active fragment thereof) and the nucleic acid encoding an IL-12p40 polypeptide can encode a full length human IL-12p40 polypeptide sequence (or a biologically active fragment thereof). In some cases, an IL-12p35 polypeptide sequence and an IL-12p40 polypeptide sequence can be from a different species from a mammal to be treated by administering one or more recombinant vaccinia viruses including nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence. For example, when a mammal to be treated by administering one or more recombinant vaccinia viruses including nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence is a human, the nucleic acid encoding a membrane-bound IL-12p35 polypeptide can encode a full length mouse IL-12p35 polypeptide sequence (or a biologically active fragment thereof) and the nucleic acid encoding an IL-12p40 polypeptide can encode a full length mouse IL-12p40 polypeptide sequence (or a biologically active fragment thereof).

In some cases, an IL-12p35 polypeptide sequence of a membrane-bound IL-12 polypeptide can be an amino acid sequence with at least 80% sequence identity (e.g., about 82% sequence identity, about 85% sequence identity, about 88% sequence identity, about 90% sequence identity, about 93% sequence identity, about 95% sequence identity, about 97% sequence identity, about 98% sequence identity, about 99% sequence identity, or 100% sequence identity) to the amino acid sequence set forth in SEQ ID NO:2, SEQ ID NO:6, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, or SEQ ID NO:55, and an IL-12p40 polypeptide sequence of a membrane-bound IL-12 polypeptide can be an amino acid sequence with at least 80% sequence identity (e.g., about 82% sequence identity, about 85% sequence identity, about 88% sequence identity, about 90% sequence identity, about 93% sequence identity, about 95% sequence identity, about 97% sequence identity, about 98% sequence identity, about 99% sequence identity, or 100% sequence identity) to the amino acid sequence set forth in SEQ ID NO:4, SEQ ID NO:8, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, or SEQ ID NO:65.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\Bl2seq c:\seq1.txt -j c:\seq2.txt -p blastn -o c:\output.txt -q -1-r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq c:\seg1.txt -j c:\seg2.txt -p blastp -o c:\output.txt. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical nucleotide or amino acid residue is presented in both sequences. A matched position refers to a position in which identical amino acid occur at the same position in aligned sequences. The percent sequence identity is determined by dividing the number of matches by the length of the sequence set forth in the identified sequence (e.g., SEQ ID NO:2, SEQ ID NO:6, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55; SEQ ID NO:4, SEQ ID NO:8, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, or SEQ ID NO:65), followed by multiplying the resulting value by 100. For example, an amino acid sequence that has 220 matches when aligned with the sequence set forth in SEQ ID NO:2 is 93.2 percent identical to the sequence set forth in SEQ ID NO:2 (i.e., 220±236×100=93.2). It is noted that the percent sequence identity value is rounded to the nearest tenth. For example, 75.1, 75.2, 75.3, and 75.4 is rounded down to 75, while 75.5, 75.6, 75.7, 75.8, and 75.9 is rounded up to 76. It also is noted that the length value will always be an integer.

The membrane anchoring polypeptide sequence of a membrane-bound IL-12 polypeptide described herein can be any appropriate membrane anchoring polypeptide sequence. As used herein, a “membrane anchoring polypeptide sequence” can be a polypeptide sequence that can associate with a cell membrane (e.g., a lipid bilayer of a cell membrane). When a membrane anchoring polypeptide sequence is attached as a fusion protein to an IL-12p35 polypeptide sequence and/or an IL-12p40 polypeptide sequence, the membrane anchoring polypeptide sequence can tether a IL-12 polypeptide sequence to the cell membrane. In some cases, a membrane anchoring polypeptide sequence can be derived from a polypeptide (e.g., can be a fragment of a polypeptide such as a C-terminal fragment) that includes a post translational modification such as a glycosylphosphatidylinositol (GPI) modification (e.g., a GPI anchor). When a membrane anchoring polypeptide sequence is a polypeptide containing a GPI anchor, the polypeptide containing the GPI anchor can be derived from any appropriate polypeptide. Examples of polypeptides from which a membrane anchoring polypeptide sequence can be obtained include, without limitation, a CD16b polypeptide (e.g., a human CD16b polypeptide), an alkaline phosphatase polypeptide, a CD58 polypeptide, a CD14 polypeptide, a NCAM-120 polypeptide, a TAG-1 polypeptide, a CD24 polypeptide, a CD55 polypeptide, a CD56 polypeptide, a C8-binding protein polypeptide, an acetylcholine esterase polypeptide, and a CD59 polypeptide. In some cases, polypeptides containing a GPI anchor as described elsewhere (see, e.g., Ferguson et al., “Chapter 11: Glycosylphosphatidyl Anchors” in Glycobiology, 2nd Edition, Varki et al., editors, Cold Spring Harbor Press, 2009) can be used as a membrane anchoring polypeptide sequence to create a membrane-bound IL-12 polypeptide described herein. In some cases, a membrane anchoring polypeptide sequence of a membrane-bound IL-12 polypeptide described herein can be derived from a CD16b polypeptide. A membrane anchoring polypeptide sequence derived from a CD16b polypeptide can have any appropriate sequence. For example, a membrane anchoring polypeptide sequence derived from a CD16b polypeptide can have, or can be encoded by, a sequence set forth in, for example, National Center for Biotechnology Information (NCBI) Accession No: BC128562.1. Exemplary membrane anchoring polypeptide sequences derived from a CD16b polypeptide that can be used as described herein can include, without limitation, SFSPPGYQVSFCLVMVLLFA (SEQ ID NO:39) and SSFSPPGYQVSFCLVMVLLFAVDTGLYFSVKTNI (SEQ ID NO:40). Other examples of amino acid sequences that can be used as a membrane anchoring polypeptide sequence to obtain a membrane-bound IL-12 polypeptide described herein include, without limitation, those set forth in Table 1.

TABLE 1
Exemplary membrane anchoring
polypeptide sequences.

| Example | Sequence | SEQ ID |
|---|---|---|
| # | (polypeptide derived from) | NO: |
| 1 | CLEPYTACDLAPRAGTTDAAHPGPSVVPALLPLL | 41 |
| | AGTLLLLGTATAP (a PLAP polypeptide) | |
| 2 | TTCIPSSGHSRHRYALIPIPLAVITTCIVLYMNG | 42 |
| | ILKCDRKPDRTNSN (a CD58 poly- | |
| | peptide) | |
| 3 | PNKGSGTTSGTTRLLSGHTCFTLTGLLGTLVTMG | 43 |
| | LLT (a CD55 polypeptide) | |
| 4 | YTVSNMATIAVVVDLGAVAIIGAVVAFVMNRR | 44 |
| | (a Q7(b) polypeptide) | |
| 5 | LQCYNCPNPTADCKTAVNCSSDFDACLITKAGLQ | 45 |
| | VYNKCWKFEHCNFNDVTTRLRENELTYYCCKKDL | |
| | CNFNEQLEN (a CD59 polypeptide) | |

In some cases, a membrane-bound IL-12 polypeptide can include a linker (e.g., a linker between an IL-12 polypeptide sequence and a membrane anchoring polypeptide sequence). A linker can be a polypeptide linker. When a linker is a polypeptide linker, the polypeptide linker can be any appropriate length (e.g., can include any appropriate number of amino acids). For example, a polypeptide linker can include from about one amino acid to about 50 amino acids in length. Examples of linkers that can be present in a membrane-bound IL-12 polypeptide described herein include, without limitation, a (G₄S)₃ linker (e.g., GGGGSGGGGSGGGGS; SEQ ID NO:13), an A(EA₃K)₄AAA linker (e.g., AEAAAKEAAAKEAAAKEAAAKAAA; SEQ ID NO:14), a (Gly)₆ linker (e.g., GGGGGG; SEQ ID NO:15), and a (Gly)₈ linker (e.g., GGGGGGGG; SEQ ID NO:16). In some cases, a linker can be a flexible linker. In some cases, a linker can be a rigid linker. Other examples of linkers that can be used within a membrane-bound IL-12 polypeptide described herein include, without limitation, those set forth in Table 2.

TABLE 2
Exemplary linker sequences.

| Sequence | SEQ ID NO: |
|---|---|
| (EAAAK)n (n = 1-3) | 17 |
| A(EAAAK)₄ALEA(EAAAK)₄A | 18 |
| (GGGGS)n (n = 1, 2, 4) | 19 |
| AEAAAKEAAAKA | 20 |
| PAPAP | 21 |
| GGSGGSGGSGGSGGSGGSGG | 22 |

In some cases, a membrane-bound IL-12 polypeptide can include an IL-12 polypeptide sequence, a polypeptide linker sequence, and a polypeptide sequence containing a GPI anchor. For example, a membrane-bound IL-12 polypeptide can include a full length human IL-12 polypeptide sequence (or a biologically active fragment thereof) and a human CD16b polypeptide sequence containing a GPI anchor that are connected via a (G₄S)₃ (SEQ ID NO:13) linker. An exemplary amino acid sequence of a membrane-bound IL-12 polypeptide that includes an IL-12 polypeptide sequence and a human CD16b polypeptide sequence containing a GPI anchor connected via a (G₄S)₃ linker is set forth in SEQ ID NO:10.

In some cases, a membrane-bound IL-12 polypeptide can include an IL-12 polypeptide sequence, a polypeptide linker sequence, and a polypeptide sequence containing a GPI anchor. For example, a membrane-bound IL-12 polypeptide can include an IL-12 polypeptide sequence and a human CD16b polypeptide sequence containing a GPI anchor that are connected via an A(EA₃K)₄AAA (SEQ ID NO:14) linker. An exemplary amino acid sequence of a membrane-bound IL-12 polypeptide that includes an IL-12 polypeptide sequence and a human CD16b polypeptide sequence containing a GPI anchor connected via an A(EA₃K)₄AAA linker is set forth in SEQ ID NO:12.

In some cases, nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence can include one or more regulatory elements (e.g., to regulate expression of the amino acid chain). In some cases, a regulatory element can be specific for a vaccinia virus. Examples of regulatory elements that can be included in nucleic acid encoding a membrane-bound IL-12 polypeptide described herein include, without limitation, promoters (e.g., constitutive promoters, tissue/cell-specific promoters, and inducible promoters such as chemically-activated promoters and light-activated promoters), and enhancers. For example, nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence membrane-bound IL-12 polypeptide can be operably linked to a promoter (e.g., a promoter specific for vaccinia viruses) such that the promoter can regulate expression of the membrane-bound IL-12 polypeptide. Examples of promoters that can be included in a nucleic acid encoding an IL-12p35 polypeptide and/or nucleic acid encoding an IL-12p40 polypeptide where at least one of (or only one of) the IL-12p35 polypeptide and the IL-12p40 polypeptide includes a membrane anchoring polypeptide sequence membrane-bound IL-12 polypeptide described herein include, without limitation, a p7.5 e/1 promoter and a pSe/1 promoter. In some cases where a single nucleic acid sequence can encode both an IL-12p35 polypeptide (or a biologically active fragment thereof) and an IL-12p40 polypeptide (or a biologically active fragment thereof) where at least one of (or only one of) the IL-12p35 polypeptide and the IL-12p40 polypeptide includes a membrane anchoring polypeptide sequence, the nucleic acid encoding an IL-12p35 polypeptide sequence can be operably linked to a promoter, and nucleic acid encoding an IL-12p40 polypeptide sequence can be operably linked to a promoter (e.g., pSe/1 promoter), and the nucleic acid encoding an IL-12p35 polypeptide and nucleic acid encoding an IL-12p40 polypeptide can be separated by an internal ribosome entry site (IRES) such that the single nucleic acid sequence can encode both an IL-12p35 polypeptide and an IL-12p40 polypeptide. In some cases where nucleic acid encoding a IL-12p35 polypeptide sequence (or a biologically active fragment thereof) and nucleic acid encoding a IL-12p40 polypeptide sequence (or a biologically active fragment thereof) where at least one of (or only one of) the IL-12p35 polypeptide and the IL-12p40 polypeptide includes a membrane anchoring polypeptide sequence, the nucleic acid encoding an IL-12p35 polypeptide sequence can be operably linked to a first regulatory element (e.g., a first promoter) and the nucleic acid encoding an IL-12p40 polypeptide sequence can be operably linked to a second regulatory element (e.g., a second promoter different from the first promoter).

In some cases, a recombinant vaccinia virus described herein (e.g., recombinant vaccinia viruses having oncolytic anti-cancer activity) can include one or more modifications to one or more nucleic acids encoding a polypeptide (or a fragment thereof) and/or one or more viral elements of the vaccinia virus genome. The one or more modifications can be any appropriate modification. Examples of modifications that can be made to a nucleic acid encoding a polypeptide or to a viral element include, without limitation, insertions, deletions, substitutions, and mutations.

In some cases, a recombinant vaccinia virus described herein (e.g., a recombinant vaccinia virus having oncolytic anti-cancer activity) also can include one or more additional nucleic acid insertions (e.g., nucleic acid insertions other than nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence). A nucleic acid insertion can be nucleic acid encoding any appropriate polypeptide. In some cases, a nucleic acid insertion can encode a detectable label. Examples of detectable labels that can be encoded by nucleic acid in a recombinant vaccinia virus described herein include, without limitation, fluorophores (e.g., yellow fluorescent protein (YFP), GFP, mCherry, and mBFP) and enzymes (e.g., luciferase, DNAses, and proteases). For example, a recombinant vaccinia virus described herein can include nucleic acid encoding a detectable label such that, when the recombinant vaccinia virus infects a cell, the infected cell can express the detectable label. In some cases, the expression of a detectable label can be used to confirm infection of a cell (e.g., to confirm infection of a cell in vivo). In some cases, the expression of a detectable label can be used to monitor the location of an infected cell (e.g., to monitor the location of an infected cell in vivo).

In some cases, a nucleic acid insertion can encode a cytokine (e.g., a cytokine other than IL-12). Examples of cytokines that can be encoded by nucleic acid in a recombinant vaccinia virus described herein include, without limitation, an IL-1 polypeptide (e.g., an IL-113 polypeptide), an IL-2 polypeptide, an IL-3 polypeptide, an IL-4 polypeptide, an IL-5 polypeptide, an IL-6 polypeptide, an IL-7 polypeptide, an IL-8 polypeptide, an IL-9 polypeptide, an IL-10 polypeptide, an IL-11 polypeptide, an IL-13 polypeptide, an IL-15 polypeptide, an IL-17 polypeptide, an IL-18 polypeptide, an IL-21 polypeptide, an IL-23 polypeptide, an IL-24 polypeptide, an IL-27 polypeptide, a C-X-C motif chemokine 11 (CXCL11) polypeptide, a chemokine (C-C motif) ligand 5 (CCLS) polypeptide, an interferon (IFN) polypeptide (e.g., an IFN-alpha polypeptide, an IFN-alpha2 polypeptide, an IFN-beta polypeptide, and an IFN-gamma polypeptide), a tumor necrosis factor (TNF) polypeptide (e.g., a TNF-alpha polypeptide, and a TNF-beta polypeptide), and a granulocyte macrophage colony-stimulating factor (GM-CSF) polypeptide. For example, a recombinant vaccinia virus described herein can include nucleic acid encoding a cytokine (e.g., a cytokine other than IL-12) such that, when the recombinant vaccinia virus infects a cell, the infected cell can express (e.g., can express and secrete) the cytokine. In some cases, when a nucleic acid insertion into a virus provided herein encodes a cytokine other than an IL-12 cytokine, that cytokine can be designed to be membrane bound in a manner as described herein for an IL-12 polypeptide sequence. For example, a vaccinia virus can be designed to include nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence and nucleic acid encoding an IL-4 polypeptide, an IL-18 polypeptide, and/or an IL-1β polypeptide.

In some cases when a recombinant vaccinia virus described herein (e.g., a recombinant vaccinia virus having oncolytic anti-cancer activity) also includes one or more additional nucleic acid encoding a polypeptide (e.g., nucleic acid encoding a polypeptide other than an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence), the one or more additional nucleic acid insertions encoding a polypeptide can include one or more regulatory elements (e.g., to regulate expression of the amino acid chain). In some cases, a regulatory element can be specific for a vaccinia virus. Examples of regulatory elements that can be included in nucleic acid encoding a polypeptide other than an IL-12p35 polypeptide and an IL-12p40 polypeptide where at least one of (or only one of) the IL-12p35 polypeptide and the IL-12p40 polypeptide includes a membrane anchoring polypeptide sequence include, without limitation, promoters (e.g., constitutive promoters, tissue/cell-specific promoters, and inducible promoters such as chemically-activated promoters and light-activated promoters) and enhancers. For example, an additional nucleic acid encoding a polypeptide can be operably linked to a promoter (e.g., a promoter specific for vaccinia viruses) such that the promoter can regulate expression of the polypeptide. Examples of promoters that can be included in a nucleic acid encoding a polypeptide other than an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence described herein include, without limitation, a p7.5 e/1 promoter and a pSe/1 promoter. In some cases, nucleic acid encoding a polypeptide (e.g., nucleic acid encoding a polypeptide other than an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence) can be under the control of the same regulatory element(s) as the nucleic acid encoding a membrane-bound IL-12 polypeptide. In some cases, nucleic acid encoding a polypeptide (e.g., nucleic acid encoding a polypeptide other than an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence) can be under the control of a different regulatory element(s) from the nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence.

In some cases, a recombinant vaccinia virus described herein (e.g., recombinant vaccinia viruses having oncolytic anti-cancer activity) can include a vaccinia virus genome containing one or more nucleic acid deletions. A nucleic acid deletion can be any appropriate nucleic acid deletion. A nucleic acid deletion can be a full deletion (e.g., deletion of the entire nucleic acid sequence encoding a polypeptide) or a partial deletion (e.g., deletion of one or more nucleotides within a nucleic acid encoding a polypeptide, but less than the entire nucleic acid sequence encoding that polypeptide). Examples of nucleic acids that can be deleted in a recombinant vaccinia virus described herein include, without limitation, a tk locus (e.g., nucleic acid encoding all (or part) of a TK polypeptide), a vgf locus (e.g., nucleic acid encoding all (or part) of a VGF polypeptide), a K3L locus (e.g., nucleic acid encoding all (or part) a K3L polypeptide), a A56R locus (e.g., nucleic acid encoding all (or part) a A56R polypeptide), a B18R locus (e.g., nucleic acid encoding all (or part) a B18R polypeptide), and a M2L locus (e.g., nucleic acid encoding all (or part) a M2L polypeptide). For example, a recombinant vaccinia virus described herein can include a vaccinia virus genome containing a deletion of one or more nucleotides within a nucleic acid encoding a TK polypeptide. For example, a recombinant vaccinia virus described herein can include a vaccinia virus genome containing a deletion of one or more nucleotides within a nucleic acid encoding a VGF polypeptide. For example, a recombinant vaccinia virus described herein can include a vaccinia virus genome containing a deletion of one or more nucleotides within a nucleic acid encoding a TK polypeptide and a deletion of one or more nucleotides within a nucleic acid encoding a VGF polypeptide. In some cases, when a recombinant vaccinia virus described herein (e.g., a recombinant vaccinia virus having oncolytic anti-cancer activity) can include a deletion of one or more nucleotides within a tk locus. In some cases, when a recombinant vaccinia virus described herein (e.g., a recombinant vaccinia virus having oncolytic anti-cancer activity) can include a deletion of one or more nucleotides within a tk locus and can include a deletion of one or more nucleotides within a vgf locus.

In some cases, a recombinant vaccinia virus described herein (e.g., recombinant vaccinia viruses having oncolytic anti-cancer activity) can include a vaccinia virus genome containing one or more nucleic acid substitutions. A nucleic acid substitution can be any appropriate nucleic acid substitution. A nucleic acid substitution can be a full substitution (e.g., substitution of the entire nucleic acid sequence encoding a polypeptide) or a partial substitution (e.g., substitution of one or more nucleotides within a nucleic acid encoding a polypeptide, but less than the entire nucleic acid sequence encoding that polypeptide). Examples of nucleic acids that can be substituted in a recombinant vaccinia virus described herein include, without limitation, a tk locus (e.g., nucleic acid encoding all (or part) of a TK polypeptide), a vgf locus (e.g., nucleic acid encoding all (or part) of a VGF polypeptide), a K3L locus (e.g., nucleic acid encoding all (or part) a K3L polypeptide), a A56R locus (e.g., nucleic acid encoding all (or part) a A56R polypeptide), a B18R locus (e.g., nucleic acid encoding all (or part) a B18R polypeptide), and a M2L locus (e.g., nucleic acid encoding all (or part) a M2L polypeptide). For example, a recombinant vaccinia virus described herein can include a vaccinia virus genome containing a substitution of one or more nucleotides within a nucleic acid encoding a TK polypeptide. For example, a recombinant vaccinia virus described herein can include a vaccinia virus genome containing a substitution of one or more nucleotides within a nucleic acid encoding a VGF polypeptide. For example, a recombinant vaccinia virus described herein can include a vaccinia virus genome containing a substitution of one or more nucleotides within a nucleic acid encoding a TK polypeptide and a substitution of one or more nucleotides within a nucleic acid encoding a VGF polypeptide. In some cases, when a recombinant vaccinia virus described herein (e.g., a recombinant vaccinia virus having oncolytic anti-cancer activity) includes a substitution of one or more nucleotides within a nucleic acid encoding a TK polypeptide, one or more nucleotides within the tk locus can be substituted (e.g., with nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence).

This document also provides methods for using one or more recombinant vaccinia viruses described herein (e.g., one or more recombinant vaccinia viruses having oncolytic anti-cancer activity). In some cases, a recombinant vaccinia virus provided herein can used to treat a mammal having, or at risk of having, cancer. For example, methods for treating a mammal having, or at risk of having, cancer can include administering one or more recombinant vaccinia viruses described herein to the mammal. In some cases, methods for treating a mammal having, or at risk of having, cancer can include administering nucleic acid (e.g., one or more expression vectors) encoding a recombinant vaccinia virus described herein to the mammal.

When a recombinant vaccinia virus described herein (e.g., a recombinant vaccinia virus having oncolytic anti-cancer activity) is administered to a mammal, the vaccinia virus can infect any appropriate type of cell within the mammal. Examples of cell types that can be infected by a recombinant vaccinia virus described herein include, without limitation, epithelial cells, stromal cells, dendritic cells, and activated T cells. In some cases, a cell that can be infected by a recombinant vaccinia virus described herein can be a cancer cell. In some cases, a cell that can be infected by a recombinant vaccinia virus described herein can be a stromal cell (e.g., a stromal cell present in a TME).

In some cases, one or more recombinant vaccinia viruses described herein (e.g., one or more recombinant vaccinia viruses having oncolytic anti-cancer activity) can be administered to a mammal to reduce the number of cancer cells present within a mammal, to reduce the size of a tumor present within a mammal, and/or to reduce the volume of one or more tumors present within a mammal. For example, one or more recombinant vaccinia viruses provided herein can be administered to a mammal (e.g., a human) in need thereof (e.g., a human having cancer) as described herein to reduce the number of cancer cells present within the mammal, to reduce the size of a tumor present within the mammal, and/or to reduce the volume of one or more tumors present within the mammal by, for example, 10, 20, 30, 40, 50, 60, 70, 80, 90, 95, or more percent.

In some cases, one or more recombinant vaccinia viruses described herein (e.g., one or more recombinant vaccinia viruses having oncolytic anti-cancer activity) can be administered to a mammal to increase the survival of the mammal (e.g., as compared to a mammal having, or at risk of developing, a cancer that is not administered one or more recombinant vaccinia viruses described herein). For example, one or more recombinant vaccinia viruses provided herein can be administered to a mammal (e.g., a human) in need thereof (e.g., a human having cancer) as described herein to increase the survival of the mammal by, for example, 10, 20, 30, 40, 50, 60, 70, 80, 90, 95, or more percent. For example, one or more recombinant vaccinia viruses provided herein can be administered to a mammal (e.g., a human) in need thereof (e.g., a human having cancer) as described herein to increase the survival of the mammal by, for example, about 1 month, about 2 months, about 4 months, about 6 months, about 8 months, about 10 months, about 12 months, about 14 months, about 18 months, about 20 months, about 2 years, about 3 years, about 5 years, or more.

In some cases, one or more recombinant vaccinia viruses described herein (e.g., one or more recombinant vaccinia viruses having oncolytic anti-cancer activity) can be administered to a mammal to facilitate entry of one or more T cells (e.g., activated T cells) into a TME (e.g., to increase the amount of one or more T cells in the TME) within the mammal. For example, one or more recombinant vaccinia viruses provided herein can be administered to a mammal (e.g., a human) in need thereof (e.g., a human having cancer) as described herein to recruit one or more T cells to the TME of a tumor (e.g., to increase the amount of one or more T cells in the TME of a tumor) within the mammal by, for example, 10, 20, 30, 40, 50, 60, 70, 80, 90, 95, or more percent. Examples of T cells that can be increased in a TME following administration of one or more recombinant vaccinia viruses provided herein can include, without limitation, CD4+ T cells, CD8+ T cells, and natural killer T cells.

In some cases, one or more recombinant vaccinia viruses described herein (e.g., one or more recombinant vaccinia viruses having oncolytic anti-cancer activity) can be administered to a mammal to reduce or eliminate entry of one or more T cells (e.g., suppressor T cells) into a TME (e.g., to reduce or eliminate the number of one or more T cells in the TME) within the mammal. For example, one or more recombinant vaccinia viruses provided herein can be administered to a mammal (e.g., a human) in need thereof (e.g., a human having cancer) as described herein to reduce or eliminate the number of one or more T cells in the TME of a tumor within the mammal by, for example, 10, 20, 30, 40, 50, 60, 70, 80, 90, 95, or more percent. Examples of T cells that can be reduced or eliminated in a TME of a tumor following administration of one or more recombinant vaccinia viruses provided herein can include, without limitation, Tregs, G-MDSCs, and exhausted T cells (e.g., exhausted CD8⁺ T cells).

In some cases, one or more recombinant vaccinia viruses described herein (e.g., one or more recombinant vaccinia viruses having oncolytic anti-cancer activity) can be administered to a mammal to increase the level of one or more polypeptides (e.g., cytokines) in a TME (e.g., to increase the amount of one or more cytokines in the TME) within the mammal. For example, one or more recombinant vaccinia viruses provided herein can be administered to a mammal (e.g., a human) in need thereof (e.g., a human having cancer) as described herein to increase the amount of one or more cytokines in the TME of a tumor within the mammal by, for example, 10, 20, 30, 40, 50, 60, 70, 80, 90, 95, or more percent. An example of a cytokine whose level can be increased in a tumor following administration of one or more recombinant vaccinia viruses provided herein can include, without limitation, an IFN-γ polypeptide.

In some cases, one or more recombinant vaccinia viruses described herein (e.g., one or more recombinant vaccinia viruses having oncolytic anti-cancer activity) can be administered to a mammal to decrease the level of one or more polypeptides in a cancer cell within the mammal. For example, one or more recombinant vaccinia viruses provided herein can be administered to a mammal (e.g., a human) in need thereof (e.g., a human having cancer) as described herein to decrease the level of one or more polypeptides in a cancer cell within the mammal by, for example, 10, 20, 30, 40, 50, 60, 70, 80, 90, 95, or more percent. Examples of polypeptides whose level can be decreased in a cancer cell following administration of one or more recombinant vaccinia viruses provided herein can include, without limitation, a TGF-β polypeptide, a COX-2 polypeptide, and a VEGF polypeptide.

In some cases, when one or more recombinant vaccinia viruses described herein (e.g., one or more recombinant vaccinia viruses having oncolytic anti-cancer activity) are administered to a mammal, the mammal can experience minimal to no adverse effects (e.g., as compared to a mammal having, or at risk of developing, a cancer that is administered a comparable control vaccinia virus that lacks nucleic acid encoding an IL-12p35 polypeptide and nucleic acid encoding an IL-12p40 polypeptide where at least one of (or only one of) the IL-12p35 polypeptide and the IL-12p40 polypeptide includes a membrane anchoring polypeptide sequence). Examples of adverse effects that can be experienced by a mammal in a minimal manner when one or more recombinant vaccinia viruses described herein are administered to the mammal include, without limitation, leukopenia, thrombocytopenia, and pulmonary edema.

Any appropriate mammal having a cancer can be treated as described herein (e.g., by administering one or more recombinant vaccinia viruses described herein). Examples of mammals that can have a cancer and that can be treated as described herein include, without limitation, humans, non-human primates (e.g., monkeys), horses, bovine species, porcine species, dogs, cats, mice, and rats. In some cases, a human having cancer can be treated as described herein.

A mammal having, or at risk of developing, any type of cancer can be treated as described herein (e.g., by administering one or more recombinant vaccinia viruses described herein). In some cases, a cancer can include one or more solid tumors. In some cases, a cancer can be a blood cancer. Examples of cancers that can be treated as described herein include, without limitation, colon cancer, lung cancer, prostate cancer, ovarian cancer, hepatocellular carcinoma, pancreatic cancer, kidney cancer, melanoma, brain cancer, lymphoma, myeloma, leukemias (e.g., lymphocytic leukemias and myelogenous leukemias), and breast cancer.

In some cases, methods for treating cancer described herein also can include identifying a mammal as having, or as being at risk of developing, the cancer. Examples of methods for identifying a mammal as having cancer include, without limitation, physical examination, laboratory tests (e.g., blood and/or urine), biopsy, imaging tests (e.g., X-ray, PET/CT, MRI, and/or ultrasound), nuclear medicine scans (e.g., bone scans), endoscopy, and/or genetic tests. Examples of methods for identifying a mammal as being at risk of developing a cancer include, without limitation, evaluating family histories for cancer, identifying the mammal as previously having had a cancer, and/or genetic testing. Once identified as having, or as being at risk of developing, a cancer, a mammal can be administered or instructed to self-administer one or more recombinant vaccinia viruses described herein (e.g., one or more recombinant vaccinia viruses having oncolytic anti-cancer activity).

In some cases, one or more recombinant vaccinia viruses described herein (e.g., one or more recombinant vaccinia viruses having oncolytic anti-cancer activity) can be formulated into a composition (e.g., a pharmaceutically acceptable composition) for administration to a mammal having, or at risk of developing, a cancer. For example, one or more vaccinia viruses described herein can be formulated together with one or more pharmaceutically acceptable carriers (additives), excipients, and/or diluents. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

In some cases, a composition (e.g., a pharmaceutical composition) including one or more recombinant vaccinia viruses described herein (e.g., one or more recombinant vaccinia viruses having oncolytic anti-cancer activity) can be administered to a mammal (e.g., a mammal having, or at risk of having, cancer) as a vaccine. A vaccine can be prophylactic or therapeutic.

A composition including one or more recombinant vaccinia viruses described herein (e.g., one or more recombinant vaccinia viruses having oncolytic anti-cancer activity) can be administered (e.g., can be designed for any type of administration) to a mammal having, or at risk of developing, a cancer. For example, a composition including one or more recombinant vaccinia viruses described herein can be designed for oral or parenteral (including, without limitation, a subcutaneous, intramuscular, intravenous, intradermal, intra-cerebral, intrathecal, or intraperitoneal (i.p.) injection) administration to a mammal having, or at risk of developing, a cancer. Compositions suitable for oral administration include, without limitation, liquids, tablets, capsules, pills, powders, gels, and granules. In some cases, compositions suitable for oral administration can be in the form of a food supplement. In some cases, compositions suitable for oral administration can be in the form of a drink supplement. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

A composition including one or more recombinant vaccinia viruses described herein (e.g., one or more recombinant vaccinia viruses having oncolytic anti-cancer activity) can be administered to a mammal having, or at risk of developing, a cancer in any appropriate amount (e.g., any appropriate dose). Effective amounts can vary depending on the route of administration, the age and general health condition of the subject, excipient usage, the possibility of co-usage with other therapeutic treatments such as use of other agents, and the judgment of the treating physician. An effective amount of a composition containing one or more recombinant vaccinia viruses described herein can be any amount that can treat a mammal having, or at risk of developing, a cancer as described herein without producing significant toxicity to the mammal. For example, an effective amount of recombinant vaccinia viruses described herein can be from about 1.0×10⁶ plaque forming units (PFU) to about 1.0×10¹⁰ PFU (e.g., about 2×10⁸PFU). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the cancer may require an increase or decrease in the actual effective amount administered.

A composition containing one or more recombinant vaccinia viruses described herein (e.g., one or more recombinant vaccinia viruses having oncolytic anti-cancer activity) can be administered to a mammal having, or at risk of developing, a cancer in any appropriate frequency. The frequency of administration can be any frequency that can treat a mammal having, or at risk of developing, a cancer without producing significant toxicity to the mammal. For example, the frequency of administration can be from about once every two days to about once a week, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition containing one or more recombinant vaccinia viruses described herein (e.g., one or more recombinant vaccinia viruses having oncolytic anti-cancer activity) can be administered to a mammal having, or at risk of developing, a cancer for any appropriate duration. An effective duration for administering or using a composition containing one or more recombinant vaccinia viruses described herein can be any duration that can treat a mammal having, or at risk of developing, a cancer without producing significant toxicity to the mammal. For example, the effective duration can vary from several weeks to several months, from several months to several years, or from several years to a lifetime. In some cases, the effective duration can range in duration from about 10 years to about a lifetime. Multiple factors can influence the actual effective duration used for a particular treatment. For example, an effective duration can vary with the frequency of administration, effective amount, use of multiple treatment agents, and/or route of administration.

In some cases, a composition containing one or more (e.g., one, two, three, four, five or more) recombinant vaccinia viruses described herein (e.g., one or more recombinant vaccinia viruses having oncolytic anti-cancer activity) can include the one or more vaccinia viruses as the sole active ingredient(s) in the composition effective to treat a mammal having, or at risk of developing, a cancer.

In some cases, a composition containing one or more (e.g., one, two, three, four, five or more) recombinant vaccinia viruses described herein (e.g., one or more recombinant vaccinia viruses having oncolytic anti-cancer activity) can include one or more (e.g., one, two, three, four, five or more) additional active agents (e.g., therapeutic agents) in the composition that are effective to treat a mammal having, or at risk of developing, a cancer.

In some cases, a mammal having, or at risk of developing, a cancer being treated as described herein (e.g., by administering one or more recombinant vaccinia viruses having oncolytic anti-cancer activity) also can be treated with one or more (e.g., one, two, three, four, five or more) additional therapeutic agents that are effective to treat a mammal having, or at risk of developing, a cancer. A therapeutic agent used in combination with one or more recombinant vaccinia viruses described herein can be any appropriate therapeutic agent. In some cases, a therapeutic agent can be a chemotherapeutic agent. In some cases, a therapeutic agent can be a targeted cancer drug. In some cases, a therapeutic agent can be an immunotherapy. Examples of therapeutic agents that can be used in combination with one or more recombinant vaccinia viruses described herein include, without limitation, chemokines (e.g., to improve tumor T cell infiltration of a tumor), cytokines (e.g., to reprogram immune cells), immune checkpoint inhibitors (e.g., cytotoxic T lymphocyte antigen 4 (CTLA-4) antagonists such as anti-CTLA-4 antibodies, CD28 antagonists such as anti-CD28 antibodies, programmed cell death 1 (PD-1) antagonists such as anti-PD-1 antibodies, and programmed cell death 1 ligand 1 (PD-L1) antagonists such as anti-PD-L1 antibodies), and combinations thereof. In some cases, the one or more additional therapeutic agents can be administered together with the one or more recombinant vaccinia viruses (e.g., in a composition containing one or more recombinant vaccinia viruses and containing one or more additional therapeutic agents). In some cases, the one or more (e.g., one, two, three, four, five or more) additional therapeutic agents can be administered independent of the one or more recombinant vaccinia viruses. When the one or more additional therapeutic agents are administered independent of the one or more recombinant vaccinia viruses, the one or more recombinant vaccinia viruses can be administered first, and the one or more additional therapeutic agents administered second, or vice versa.

In some cases, a mammal having, or at risk of developing, a cancer being treated as described herein (e.g., by administering one or more recombinant vaccinia viruses having oncolytic anti-cancer activity) also can be treated with one or more (e.g., one, two, three, four, five or more) additional therapies (e.g., therapeutic interventions) that are effective to treat a mammal having, or at risk of developing, a cancer. Examples of therapies that can be used in combination with one or more recombinant vaccinia viruses described herein include, without limitation, surgery, radiation therapy, bone marrow transplant, cryoablation, radiofrequency ablation, chemotherapy, and combinations thereof. In some cases, the one or more additional therapies that are effective to treat a mammal having, or at risk of developing, a cancer can be performed at the same time as the administration of the one or more recombinant vaccinia viruses. In some cases, the one or more additional therapies that are effective to treat a mammal having, or at risk of developing, a cancer can be performed before and/or after administration of one or more recombinant vaccinia viruses provided herein.

In some cases, methods for treating a mammal (e.g., a human) having, or at risk of developing, a cancer as described herein (e.g., by administering one or more recombinant vaccinia viruses having oncolytic anti-cancer activity) also can include monitoring the mammal being treated. For example, when treating a mammal having a cancer, the mammal can be monitored for any change in the number of cancer cells within the mammal (e.g., a change in the size of a tumor within the mammal and/or a change in the volume of a tumor within the mammal). Any appropriate method (e.g., physical examination, laboratory tests (e.g., blood and/or urine), biopsy, imaging tests (e.g., X-ray, PET/CT, MRI, and/or ultrasound), nuclear medicine scans (e.g., bone scans), and/or endoscopy) can be used to monitor the number of cancer cells within a mammal. When treating a mammal at risk of developing a cancer, the mammal can be monitored for the development of cancer. Any appropriate method (e.g., physical examination, laboratory tests (e.g., blood and/or urine), biopsy, imaging tests (e.g., X-ray, PET/CT, MRI, and/or ultrasound), nuclear medicine scans (e.g., bone scans), and/or endoscopy) can be used to monitor a mammal for development of a cancer. In some cases, methods described herein also can include monitoring a mammal being treated as described herein for toxicity. The level of toxicity, if any, can be determined by assessing a mammal's clinical signs and symptoms before and after administering a known amount of one or more recombinant vaccinia viruses described herein. It is noted that the effective amount of one or more recombinant vaccinia viruses described herein administered to a mammal can be adjusted according to a desired outcome as well as the mammal's response and level of toxicity.

The invention will be further described in the following examples, which do not limit the scope of the invention described in the claims.

### EXAMPLES

### Example 1: Oncolytic Vaccinia Virus Delivering Tethered IL-12 Enhances Antitumor Effects with Improved Safety

Mice and cell Lines

Female C57BL/6 (B6 in short) and BalB/c mice were purchased from The Jackson Laboratory (Bar Harbor, Me.) and housed in specific pathogen-free conditions. All animal studies were approved by the University of Pittsburgh Institutional Animal Care and Use Committee. Mouse colon cancer MC38-luc, colon cancer CT26-luc, and mesothelioma AB12-luc cells were generated by the infection of parental tumor cells with firefly luciferase-carrying lentivirus and antibiotic blasticidin selection. Normal African green monkey kidney fibroblast CV1, mouse melanoma B16, and Lewis lung cancer cells were obtained from American Type Culture Collection. Primary T cells were grown in RPMI-1640 supplemented with 10% fetal bovine serum (FBS), 1 mM sodium pyruvate, 2 mM L-glutamine, and 1× penicillin/streptomycin (Invitrogen, Carlsbad, Calif.). Other cell lines were grown in Dulbecco's Modified Eagle's medium (DMEM) supplemented with 10% FBS, 2 mM L-glutamine, and 1× penicillin/streptomycin in a 37° C., 5% CO₂ incubator.

### Virus Generation

VSC20, a vgf gene-deleted Western Reserve strain vaccinia virus, was used as the parental virus for homologous recombination. Murine IL-12p35 and IL-12p40 cDNAs were inserted into shuttle vectors pCMS1-IRES, pCMS1-IRES-FG, or pCMS1-IRES-RG to get shuttle plasmids pCMS1-IL-12p35-IRES-IL-12p40, pCMS1-IL-12p35-IRES-IL-12p40-FG, or pCMS1-IL-12p35-IRES-IL-12p40-RG, respectively. The primers for plasmid cloning based on polymerase chain reaction (PCR) are listed in Table 3. All these shuttle vectors were used for homologous recombination of murine IL-12 variants into the tk locus of the vaccinia viral genome. To make the new viruses vvDD-IL-12, vvDD-IL-12-FG, and vvDD-IL-12-RG, CV-1 cells were infected with VSC20 at a MOI of 0.1 and then transfected with the shuttle plasmids, resulting in virus mixture. Selection of the new recombinant viruses was based on expression of yellow fluorescent protein in CV1 cells 24 hours post infection of relative virus mixture. vvDD-YFP, or vvDD for short, a double viral gene-deleted (tk- and vgf-) vaccinia virus carrying yfp cDNA at the tk locus was used as a control virus for this work.

TABLE 3

| | SEQ ID | PCR | Cloning | Resulting |
|---|---|---|---|---|
| Primer | NO: | Template | Plasmid | Plasmid |
| P1:5′GGCGGTCGACGAGCTCATGTGTCCTCAGAAGCTAACCATC3′ | 23 | pGEM-IL-12B | pCMSl-IRES | pCMSI-IRES-L- |
| P2:5′CCGCGTTAACCTAGGATCGGACCCTGCAGGGAACAC3′ | 24 | | | 12p40 |
| P3:5′GGCGGTCGACATGGTCAGCGTTCCAACAGCCTC3′ | 25 | pGEM-IL-12A | pCMSl-IRES- | pCMSl-IL-12p35- |
| P4:5′CGCGGGCGCGCCTCAGGCGGAGCTCAGATAGCCC3′ | 26 | | IL-12p40 | IRES-IL-12p40 |
| P1:5′GGCGGTCGACGAGCTCATGTGTCCTCAGAAGCTAACCATC3′ | 27 | pGEM-IL-12B | pCMSl-IRES- | pCMSl-IL-12p40- |
| P5:5′CCGCGGCCGGCCCGGATCGGACCCTGCAGGGAACAC3′ | 28 | | FGPI | FG |
| P1:5′GGCGGTCGACGAGCTCATGTGTCCTCAGAAGCTAACCATC3′ | 29 | pCMSl-IL- | pCMSl-IRES | pCMSI-IRES-L- |
| P6:5′GCGCGTTAACTCAAATGTTTGTCTTCACAGAG3′ | 30 | 12p40-FG | | 12p40-FG |
| P3:5′GGCGGTCGACATGGTCAGCGTTCCAACAGCCTC3′ | 31 | pGEM-IL-12A | pCMSl-IRES- | pCMSl-IL-12p35- |
| P4:5′CGCGGGCGCGCCTCAGGCGGAGCTCAGATAGCCC3′ | 32 | | IL-12p40-FG | IRES-IL-12p40- |
| | | | | FG |
| P7:5′GGCCGGCCGGCCGCTGAAGCTGCCGCAAAAGAGGCCGCTGCG | 33 | pCMSl-IRES- | pCMSl-IL- | pCMSl-IL-12p40- |
| AAGGAGGCCGCGGCTAAG3′ | | RGPI | 12p40-FG | RG |
| P6:5′GCGCGTTAACTCAAATGTTTGTCTTCACAGAG3′ | 34 | | | |
| P1:5′GGCGGTCGACGAGCTCATGTGTCCTCAGAAGCTAACCATC3′ | 35 | pCMSl-IL- | pCMSl-IRES | pCMSI-IRES-L- |
| P6:5′GCGCGTTAACTCAAATGTTTGTCTTCACAGAG3′ | 36 | 12p40-RG | | 12p40-RG |
| P3:5′GGCGGTCGACATGGTCAGCGTTCCAACAGCCTC3′ | 37 | pGEM-IL-12A | pCMSl-IRES- | pCMSl-IL-12p35- |
| P4:5′CGCGGGCGCGCCTCAGGCGGAGCTCAGATAGCCC3′ | 38 | | IL-12p40-RG | IRES-IL-12p40- |
| | | | | RG |

### Viral Replication and IL-12 Expression In Vitro

MC38-luc (3×10⁵), B16 (2×10⁵), or AB12-luc (3×10⁵) cells were seeded in 24-well plates overnight and infected with vvDD, vvDD-IL-12, vvDD-IL-12-FG, or vvDD-IL-12-RG at a MOI of 1 in 0.15 mL 2% FBS-containing-DMEM for 2 hours. 0.35 mL of 10% FBS-containing-DMEM was added to cells, and the mixture was cultured until harvest at 24 hours post-viral infection. The culture supernatants were harvested to measure IL-12 using ELISA (BD Bioscience, San Jose, Calif.), and the cell pellets were applied either to measure membrane-bound IL-12 using flow cytometry or to extract RNA to measure the viral house-keeping gene A34R to monitor viral replication and transgene IL-12 expression by RT-qPCR, respectively. To further confirm the membrane association of IL-12, the tumor cells were infected with indicated viruses at MOIs of 0.1, 1, and 5 and harvested 24 hours post-infection to measure membrane-bound IL-12 using ELISA after cleavage of PI-PLC (Sigma, P5542; 8 unit per mL).

### Cytotoxicity Assay In Vitro

Tumor cells were plated at 1.0×10⁴ (except B16 cells, which were plated at 5×10³) cells per well in 96-well plates and infected with indicated viruses the next day at different MOIs. Cell viability was determined at 48 hours after infection using CellTiter 96 Aqueous Nonradioactive Cell Proliferation Assay (Promega, Madison, Mich.) or Cell Counting Kit-8 (Boster Biological Technology, Pleasanton, Calif.).

### Primary T Cell Proliferation Assay

Splenic T cells were isolated from naïve B6 mice with a pan T cell isolation kit (Miltenyi Biotec, Auburn, Calif.) and cultured in T cell medium mentioned above containing 4 μg/mL Con A and 200 U/mL IL-2 at a density of 2×10⁶/mL for two days (0.1 mL/per well in 96-well plates). On the same day, MC38 (3×10⁵) cells were seeded in 24-well plates overnight and infected with indicated viruses at an MOI of 5 in 0.15 mL 2% FBS-containing-DMEM for 2 hours. 0.35 mL of 10% FBS-containing-DMEM was added to cells, and the mixture was cultured until harvest at 24 hours post-viral infection. The mock or virus-infected MC38 cells were harvested and treated with mitomycin C (MMC) (StressMarq Biosciences: SIH-246) (200 μg/mL) in a 37° C., 5% CO₂ incubator for two hours and washed extensively for use. The MMC-treated tumor cells were re-suspended in T cell medium at a density of 1×10⁷/mL per well, and 0.1 mL was added to T cell-containing 96-well plates if needed. For some wells, MMC-treated cells were pre-incubated with anti-mIL-12 antibody (5 μg/mL; BioLegend: #505304) for half an hour before co-culture. The proliferation of activated T cells was measured using MTT assay two days after co-culture.

### Rodent Tumor Models

B6 mice were i.p. inoculated with 5×10⁵ MC38-luc cancer cells, and BalB/c mice were i.p inoculated with 4×10⁵ AB12-luc or CT26-luc cancer cells. The mice were divided into required groups at the indicated day post-tumor cell inoculation according to tumor size based on live animal IVIS imaging, performed using a Xenogen IVIS 200 Optical In Vivo Imaging System (Caliper Life Sciences, Hopkinton, Mass.). Grouped mice were i.p. injected with indicated viruses, antibodies, the combinations, or PBS. In some experiments, anti-CD8 Ab (clone 53-6.7; Bio X Cell; 250 μg per injection), anti-CD4 Ab (clone GK1.5, Bio X Cell; 150 μg per injection), anti-NK1.1 Ab (clone PK136, Bio X Cell; 300 μg per injection), or anti-IFN-γ Ab (clone XMG1.2, Bio X Cell; 200 μg per injection) were i.p. injected into mice to deplete CD8⁺ T cells, CD4⁺ T cells, or NK1.1⁺ cells or to neutralize circulating IFN-γ, respectively. Anti-PD-1 Ab (clone RMP1-14; Bio X Cell; 200 μg per injection) was i.p. injected into mice for combination therapy. In some experiments, mice were sacrificed to harvest all the individual peritoneal tumor nodules for further analysis.

MC38-luc-tumor-bearing B6 mice treated with vvDD-IL-12-FG, which after surviving longer than 250 days, were subcutaneously challenged with 5×10⁵ MC38 or 1×10⁶ Lewis lung cancer cells per mouse. Naïve B6 mice also received the same dose of tumor challenge as a control. Subcutaneous tumor size was measured using an electric caliper in two perpendicular diameters.

### Assessment of Treatment-Related Toxicity

Mouse blood samples were collected daily from virus-treated mice and kept for two hours at room temperature, and sera were separated by centrifugation to measure IL-12 and IFN-γ using commercialized kits (BioLegend), according to the vendors' instructions. The virus-treated mice were sacrificed five days post-treatments for collection of lungs, kidneys, and livers. Wet tissues were weighed.

### Flow Cytometry

Collected tumor tissues were weighed and incubated in RPMI 1640 medium containing 2% FBS, 1 mg per mL collagenase IV (Sigma: #C5138), 0.1 mg hyaluronidase (Sigma: #H6254), and 200 U DNase I (Sigma: #D5025) at 37° C. for 1-2 hours to make single cells. In vitro virus-infected cells or single cells from tumor tissues were blocked with α-CD16/32 Ab (clone 93, eBioscience: #14-0161-85; 1:1000) and then stained with antibodies against mouse CD45 (PerCP-Cy5.5 or FITC, clone: 30-F11, BioLegend: #103132 or 103108; 1:300), CD4 (APC, clone: RM4-5, eBioscience: #17-0042-81; 1:300), Foxp3 (PE, clone: FJK-16s, eBioscience: #12-5773-82; 1:100), CD8 (PE or APC, clone: 53-6.7, eBioscience: #12-0081-85 or 17-0081-83; 1:300), PD-1 (PerCP-Cy5.5, clone: 29F.1A12, BioLegend: 135208; 1:300), TIM-3 (Biotin-TIM-3, clone RMT3-23, BioLegend: #119720; 1:300+PE-SA, eBioscience: #12-4317-87; 1:1000), TIGIT (Biotin-TIGIT, clone 1G9, BioLegend: #142113; 1:300+PE-SA, eBioscience: #12-4317-87; 1:1000), LAG-3 (PE, clone: C9B7W, BioLegend: #125208; 1:300), CD11b (PE, clone:M1/7, BioLegend: #101208; 1:300), Ly-6G (APC, clone: 1A8, eBioscience: #17-9668-82; 1:300), Ly-6C (FITC, clone: HK1.4, BioLegend: #128006; 1:300), and IL-12p40 (PE, clone: C17.8, eBioscience: #12-7123-82; 1:300). The intracellular staining kit for Foxp3 and IFN-γ staining was purchased from BioLegend. Samples were collected on a BD Accuri C6 cytometer, and data were analyzed using BD Accuri C6 cytometer software.

### RT-qPCR

Total RNA was extracted from viral-infected cells or tumor tissues using the RNeasy Kit (Qiagen, Valencia, Calif.). One microgram of RNA was used for cDNA synthesis, and 25 to 50 ng of subsequent cDNA was used to conduct mRNA expression TaqMan analysis on the StepOnePlus system (Life Technologies, Grand Island, N.Y.). All primers for the analysis were purchased from Thermo Fisher Scientific (Waltham, Mass.). Gene expression was normalized to the housekeeping gene HPRT1 and expressed as fold increase (2^{-ΔCT}), where ΔCT=CT_{(Target gene)}−CT_{(HPRT1)}.

### Statistics

Statistical analyses were performed using unpaired Student's t test (GraphPad Prism version 7). Data were presented as means±SD. Animal survival was presented using Kaplan-Meier survival curves and was statistically analyzed using a log-rank test (GraphPad Prism version 7). Values of P<0.05 were considered statistically significant, and all P values were two-sided. In the figures, standard symbols were used: * P<0.05; ** P<0.01; *** P<0.001; and **** P<0.0001.

### Results

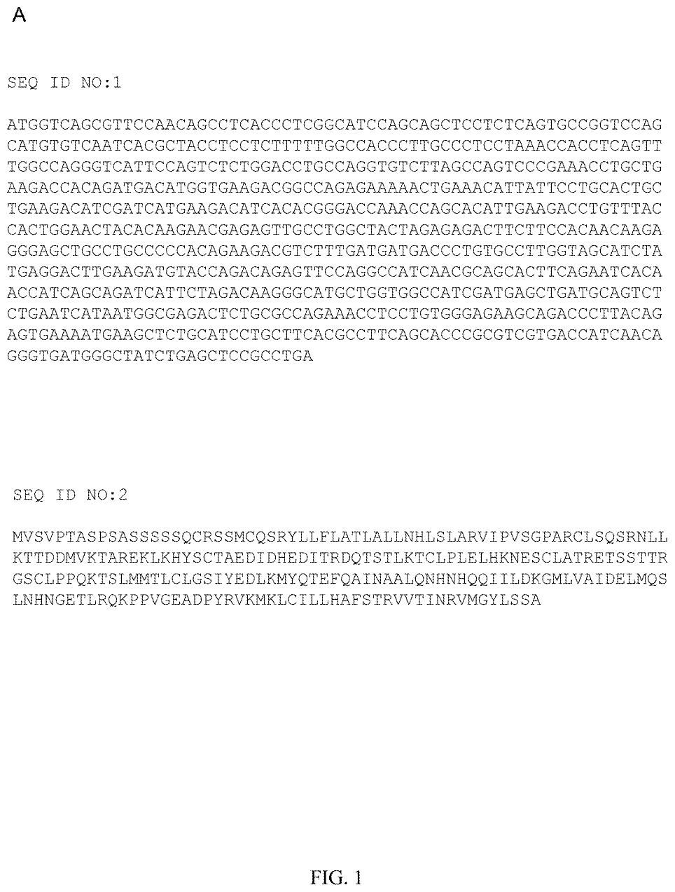

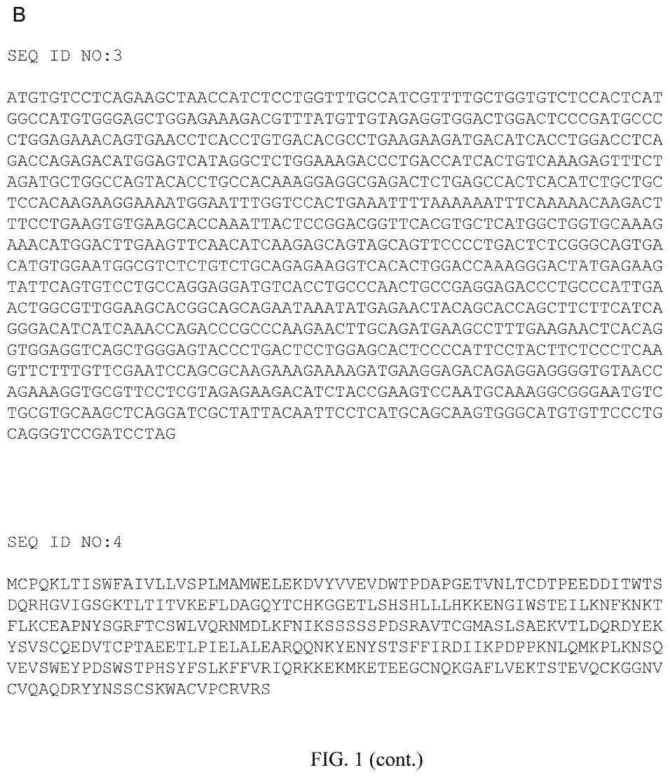

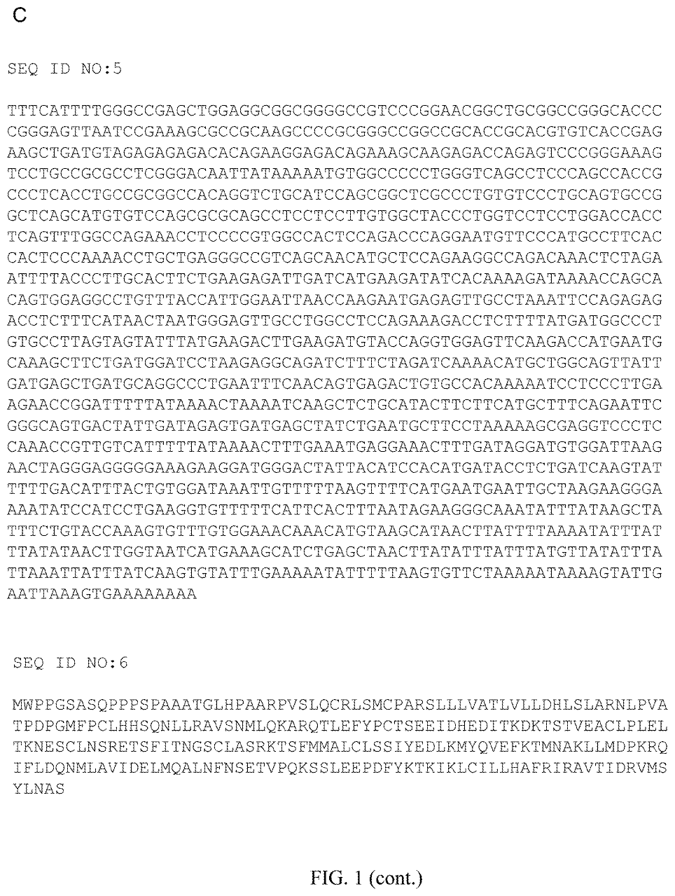

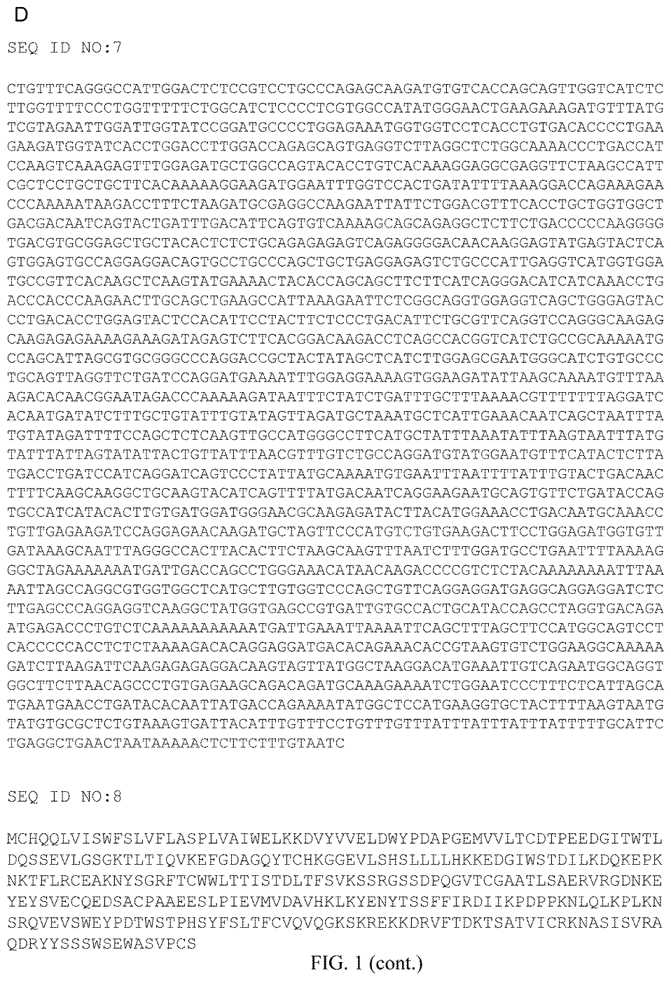

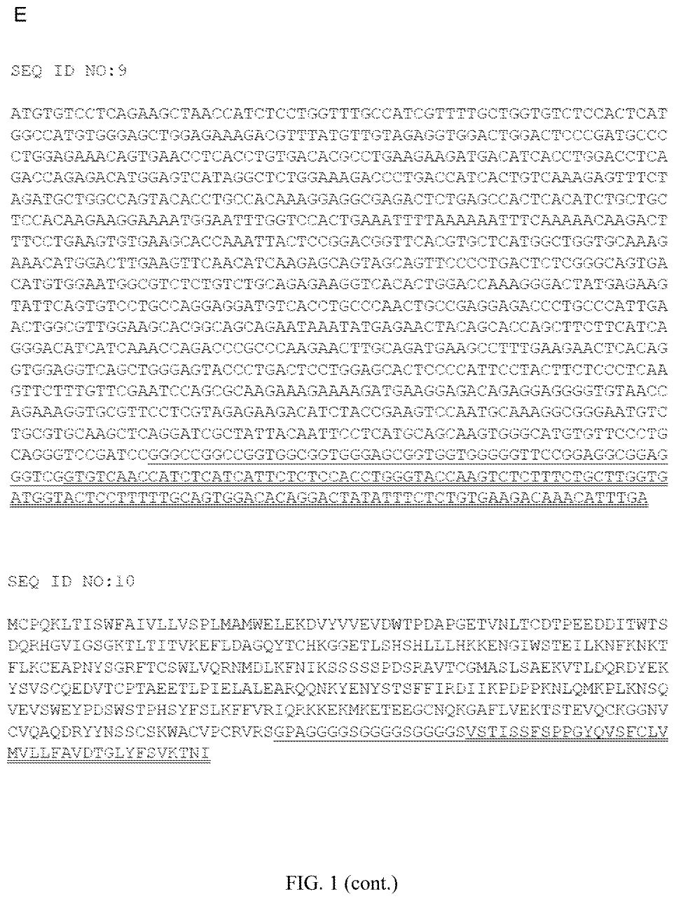

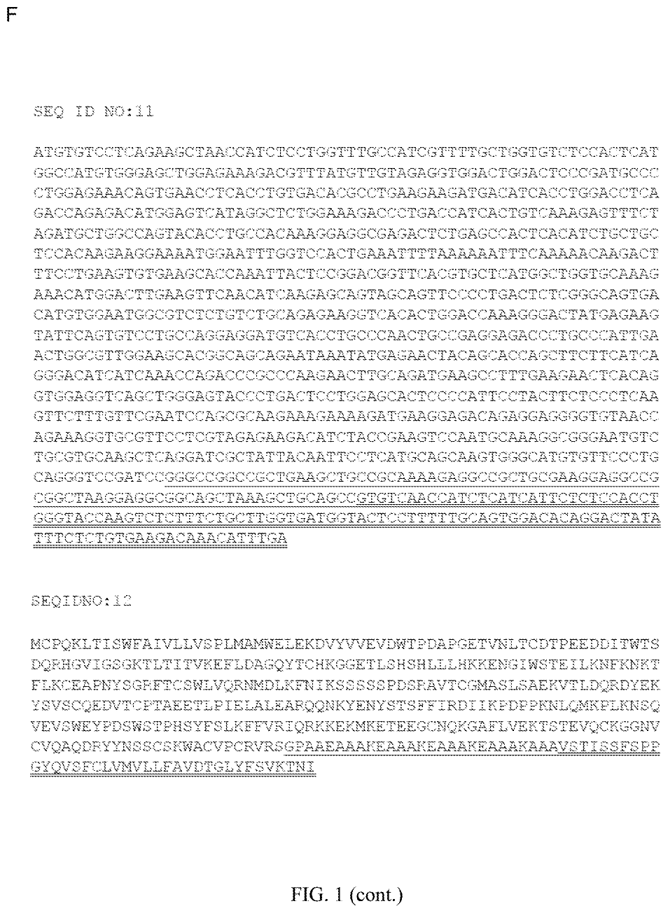

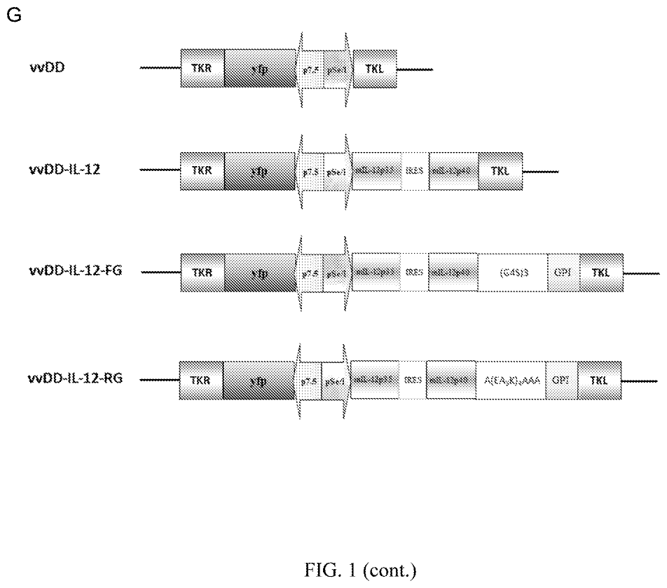

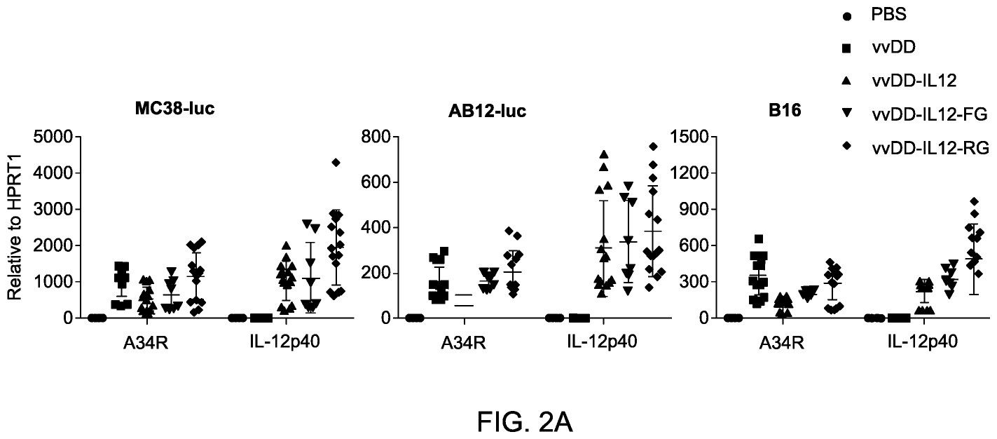

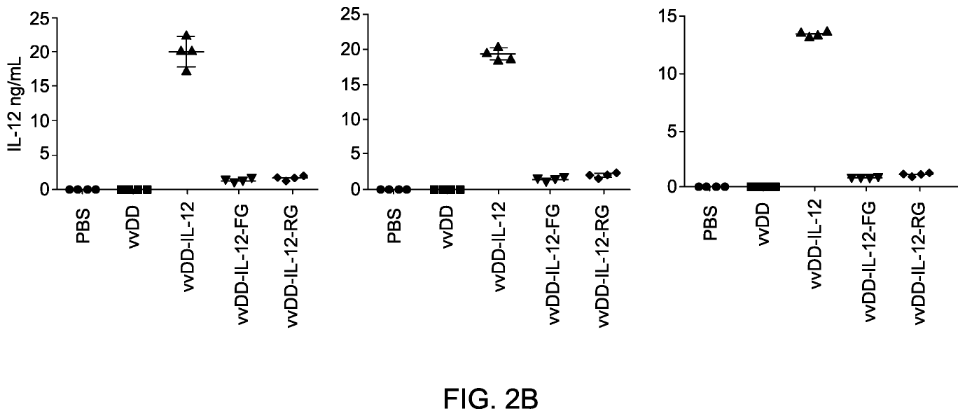

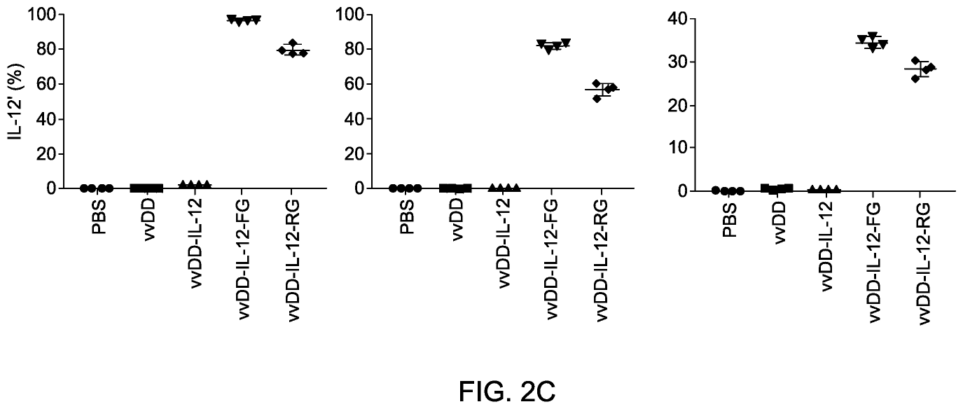

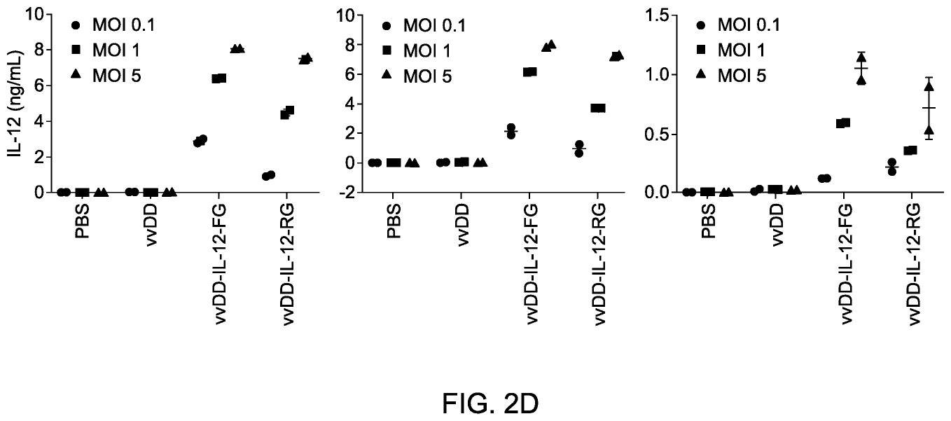

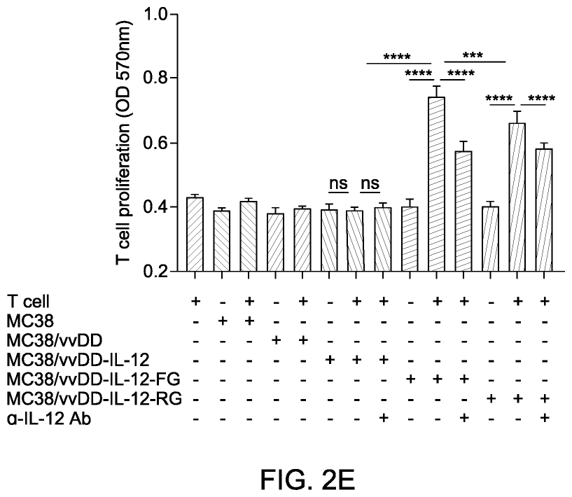

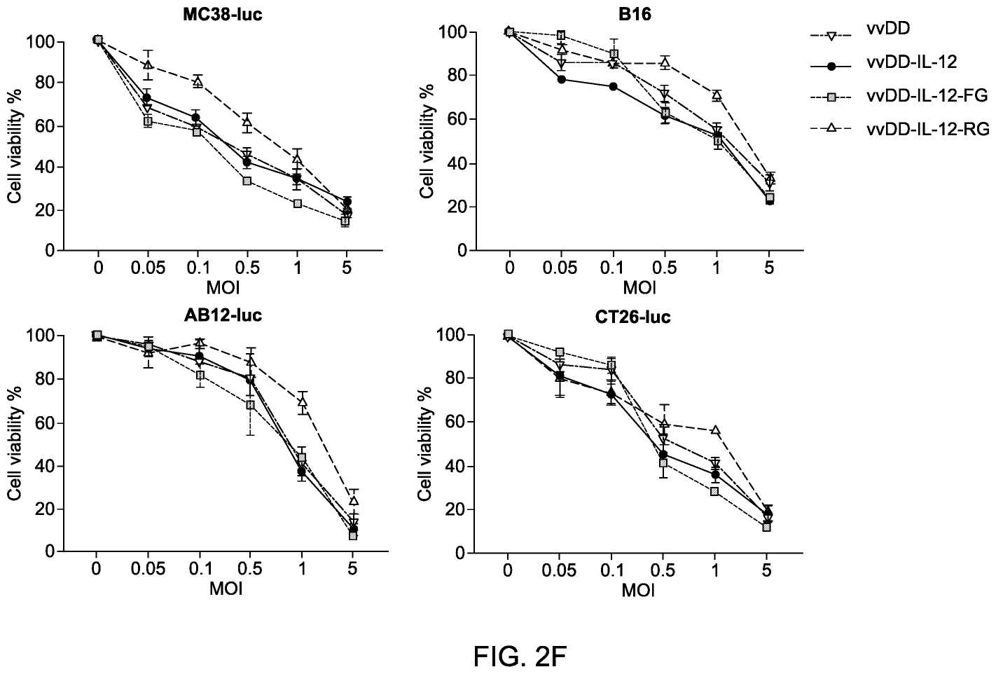

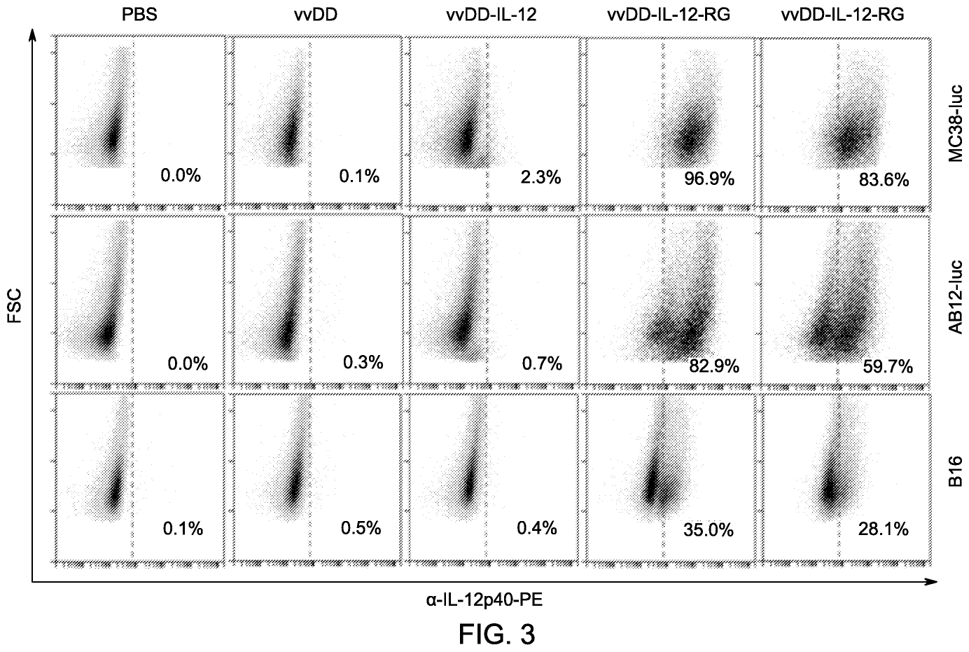

To reduce the severe toxic side effects caused by the systemic application of IL-12, vvDD, a double viral gene-deleted (tk- and vgf-) vaccinia virus, was used to deliver membrane-bound IL-12 into the tumor bed. vvDD-IL-12, vvDD-IL-12-FG, and vvDD-IL-12-RG were constructed to express secreted IL-12 (vvDD-IL-12) or membrane-bound IL-12 after infection in tumor cells (vvDD-IL-12-FG and vvDD-IL-12-RG); membrane association was achieved using the glycosylphosphatidylinositol (GPI) anchor form of human CD16b as an example. The difference between vvDD-IL-12-FG and vvDD-IL-12-RG was that in between the IL-12p40 subunit and GPI anchor, the former contained a flexible linker (G₄S)₃ (SEQ ID NO:13), and the latter contained a rigid linker A(EA₃K)₄AAA (SEQ ID NO:14) (FIG. 1). When MC38-luc, AB12-luc, and B16 cells were infected with these three IL-12-armed viruses or control virus vvDD at a multiplicity of infection (MOI) of 1, viral housekeeping gene (A34R) mRNA levels were similar in all cells infected with virus, while IL-12 mRNA levels were similar in cells infected with IL-12-armed viruses, as expected (FIG. 2A). IL-12 expression was also measured at the protein level using ELISA (enzyme-linked immunosorbent assay) and flow cytometry. The amount of IL-12 in the supernatant from vvDD-IL-12-infected tumor cells was significantly higher than the amount in the supernatants from tumor cells infected with the other constructs (FIG. 2B), while IL-12⁺ cells were significantly more prevalent in vvDD-IL-12-FG- or vvDD-IL-12-RG-infected cells (FIG. 2C; FIG. 3), showing the successful realization of membrane association by GPI anchored to one subunit of IL-12. This was further confirmed by the amount of IL-12 cleaved by phosphatidylinositol-specific phospholipase C (PI-PLC) from membrane-associated GPI-anchored IL-12. Membrane-bound IL-12 correlated with virus MOI (FIG. 2D). It was further demonstrated that the GPI-anchored IL-12 was functional in vitro. Con A-activated mouse splenic T cells were co-cultured with mitomycin C-inactivated MC38 cells, which were mock-infected or infected with the viruses overnight, and only vvDD-IL-12-FG- or vvDD-IL-12-RG-infected MC38 cells stimulated the proliferation of activated T cells, which was significantly attenuated by IL-12 antibody neutralization compared with mock-, vvDD-, or vvDD-IL-12-infected MC38 (FIG. 2E). The cytotoxicity of IL-12-armed viruses was tested in four murine tumor cells; the results showed they have similar in vitro cytotoxicity compared with the parental virus vvDD (FIG. 2F).

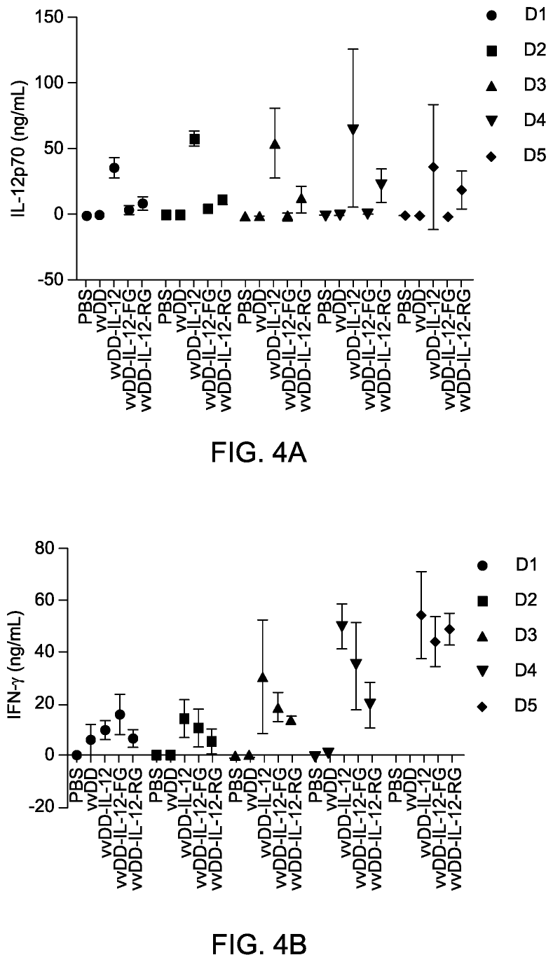

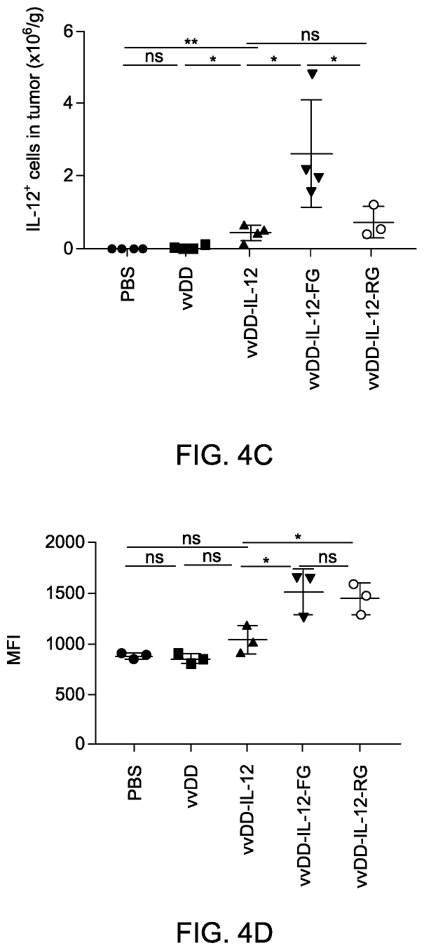

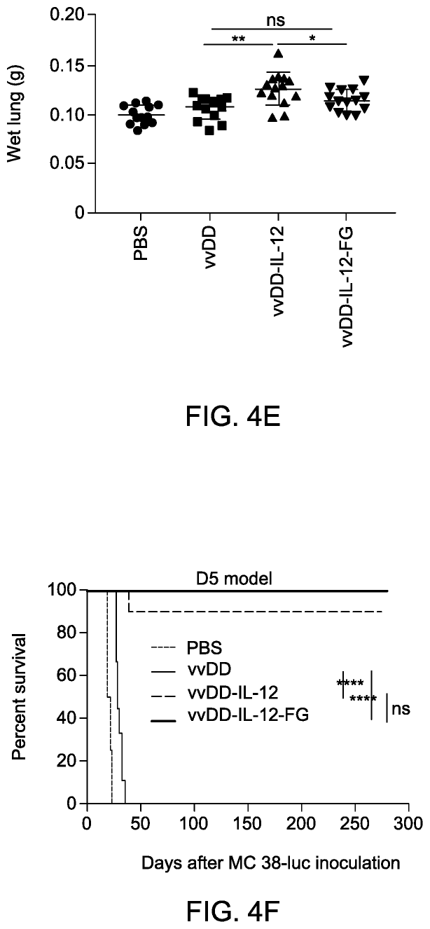

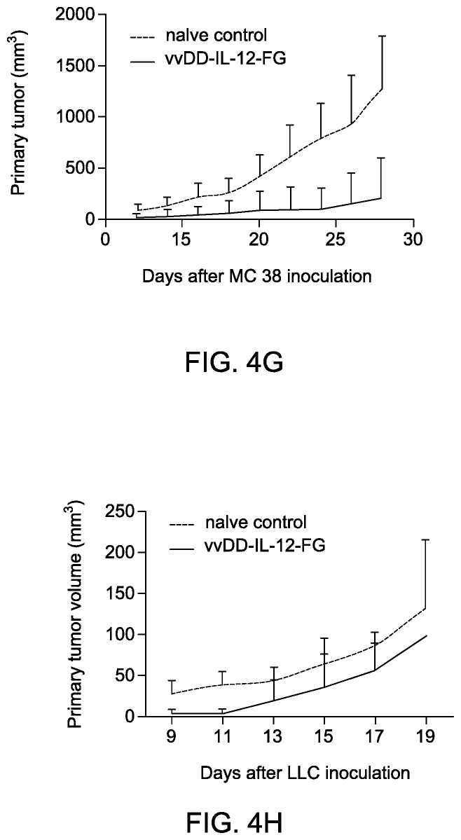

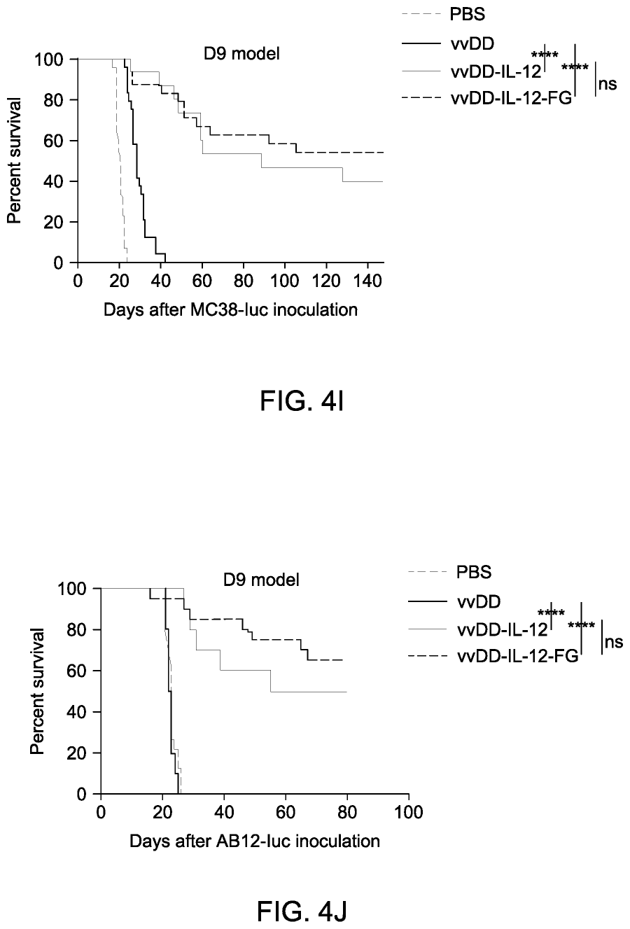

To investigate the toxicity induced by these viruses, the amount of IL-12 in mouse sera was measured, and it was found that IL-12 levels were significantly higher in sera from mice treated with vvDD-IL-12 compared to those treated with membrane-bound forms (FIG. 4A). However, IFN-γ, a main mediator of IL-12-induced effects, levels in sera were similar after IL-12-armed virus treatment (FIG. 4B), demonstrating that membrane-bound IL-12 can have a similar function with a lower risk of toxicity. The membrane association of IL-12 in vivo was next investigated using flow cytometry. There were significantly more IL-12⁺ cells in tumor tissue in mice receiving vvDD-IL-12-FG treatment than in tumor tissue of mice receiving other treatments, although the mean of fluorescence intensity was similar in mice receiving either form of tethered IL-12-armed virus treatment (FIGS. 4C and 4D). The data from both in vitro and in vivo experiments showed that membrane-bound IL-12 produced by tumor cells after vvDD-IL-12-FG infection exhibited a better capacity to tether on the cell membrane without leaking into sera (FIG. 2C and FIG. 4A) compared to membrane-bound IL-12 produced by tumor cells after vvDD-IL-12-RG infections. It was also found that only vvDD-IL-12 treatment induced pulmonary edema, as evidenced by an increase in water content in lungs after treatment (FIG. 4E). Collectively, the data demonstrated that vvDD-IL-12-FG can effectively maintain IL-12 in the TME.

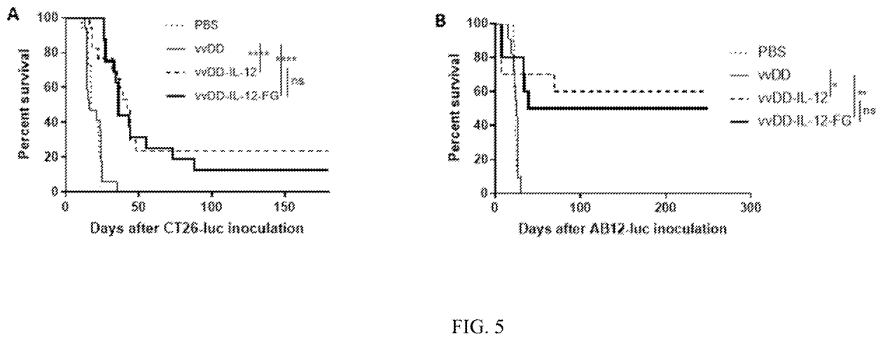

To evaluate the antitumoral efficacy of vvDD-IL-12-FG, virus was intraperitoneally (i.p.) injected at a dose of 2×10⁸PFU (plaque forming units) per mouse to treat B6 mice bearing five-day old peritoneal murine colon cancer. Survival results demonstrated that vvDD-IL-12-FG and vvDD-IL-12 treatment elicited potent anti-tumoral effects compared to phosphate buffered saline (PBS) or vvDD treatment (MC38-luc; FIG. 4F). vvDD-IL-12-FG treatment cured all mice that received the treatment, though there was no significant difference in survival between the IL-12-armed virus treatments. All the mice bearing peritoneal MC38-luc cured by vvDD-IL-12-FG treatment received a subcutaneous re-challenge of either MC38 or an irrelevant tumor control, Lewis lung cancer (LLC). MC38 tumor growth was retarded in the cured mice (FIG. 4G), but LLC was not (FIG. 4H) compared with naïve mouse control, suggesting that a systemic tumor-specific antitumor immunity was elicited. The therapeutic efficacy of vvDD-IL-12-FG was explored by applying 2×10⁸ or 1×10⁸PFU of vvDD-IL-12-FG per mouse to treat BalB/c mice bearing five-day old peritoneal murine colon cancer (CT26-luc; FIG. 5A) or murine mesothelioma (AB12-luc; FIG. 5B) with similar results. The anti-tumoral efficacy of vvDD-IL-12-FG was also evaluated in a nine-day-tumor-bearing mouse model, which is more akin to metastatic human tumors, characterized with heavier tumor burden and increased immunosuppressive factor expression in the TME (PD-1, PD-L1, CTLA-4, TGF-β, CD105, and VEGF). Both IL-12-armed virus treatments significantly improved survival compared with PBS or vvDD treatment in the nine-day-MC38 model (FIG. 4I). Similar results were obtained using a nine-day-AB12-bearing mouse model (FIG. 4J). Occasionally, a few mice that received vvDD-IL-12 treatment, but not vvDD-IL-12-FG treatment, died earlier than those that received PBS treatment (FIG. 4J), implying that the vvDD-IL-12 resulted in IL-12 induced toxicity.

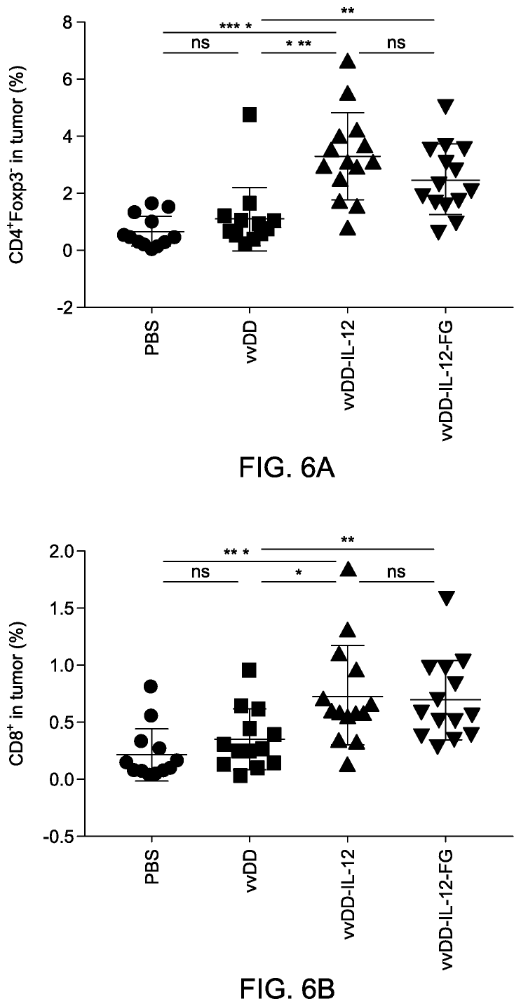

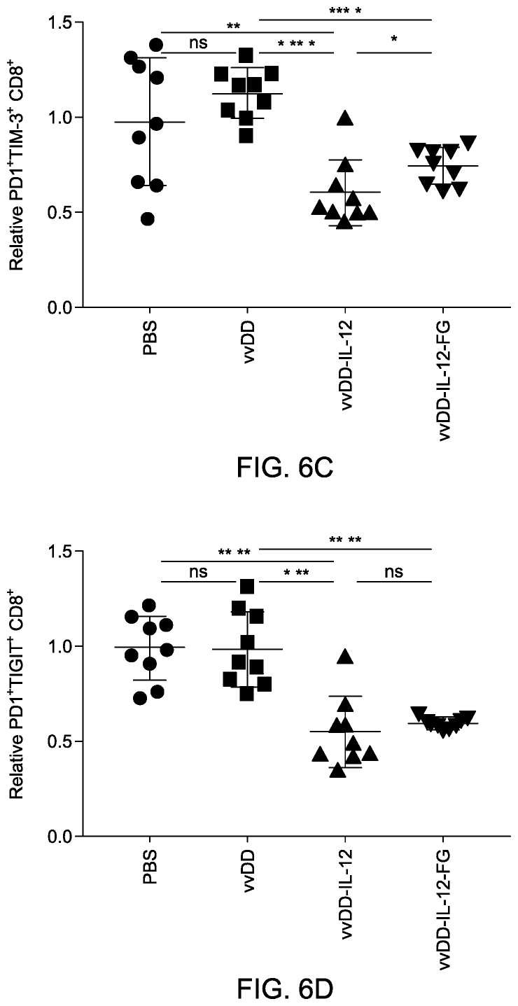

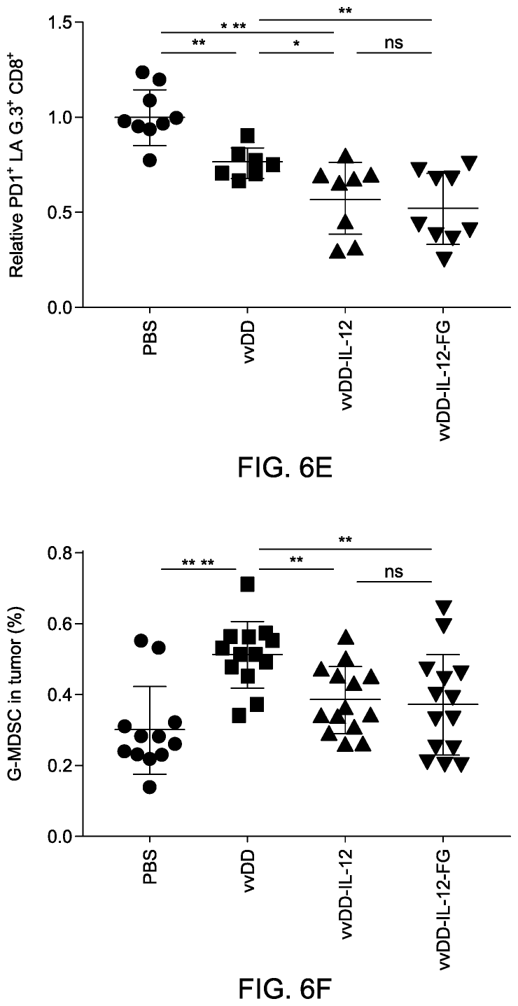

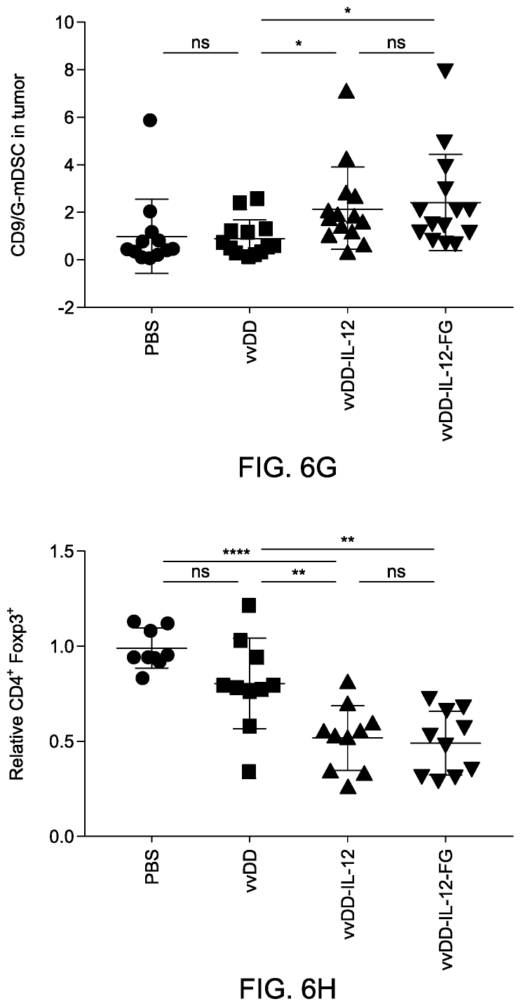

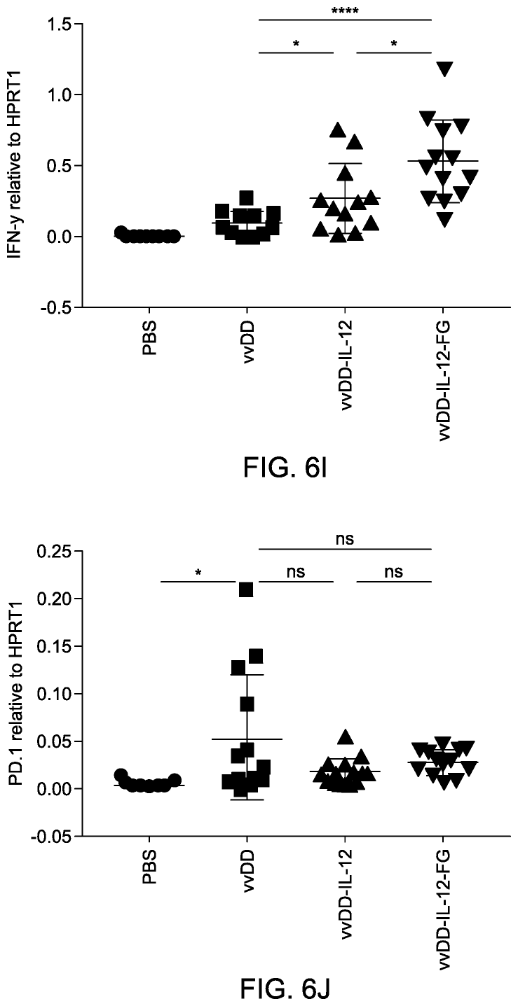

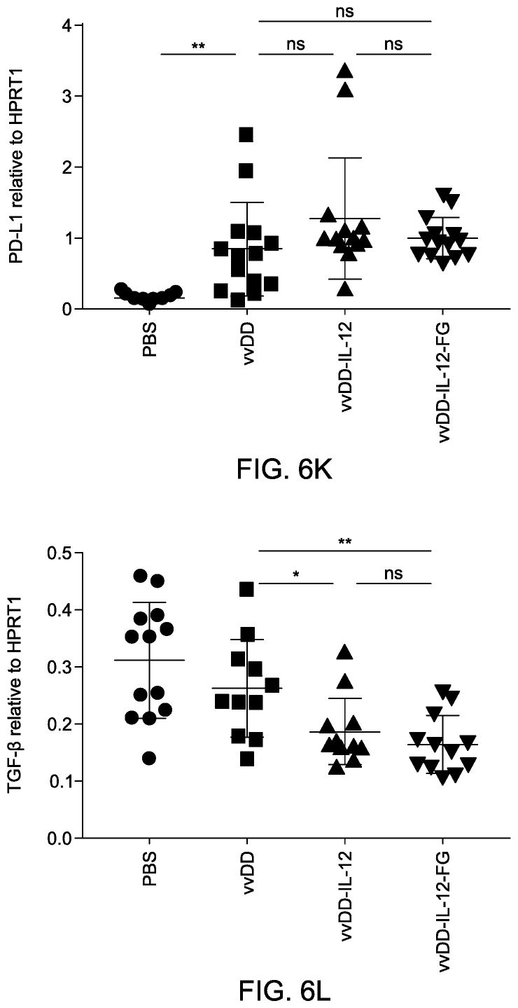

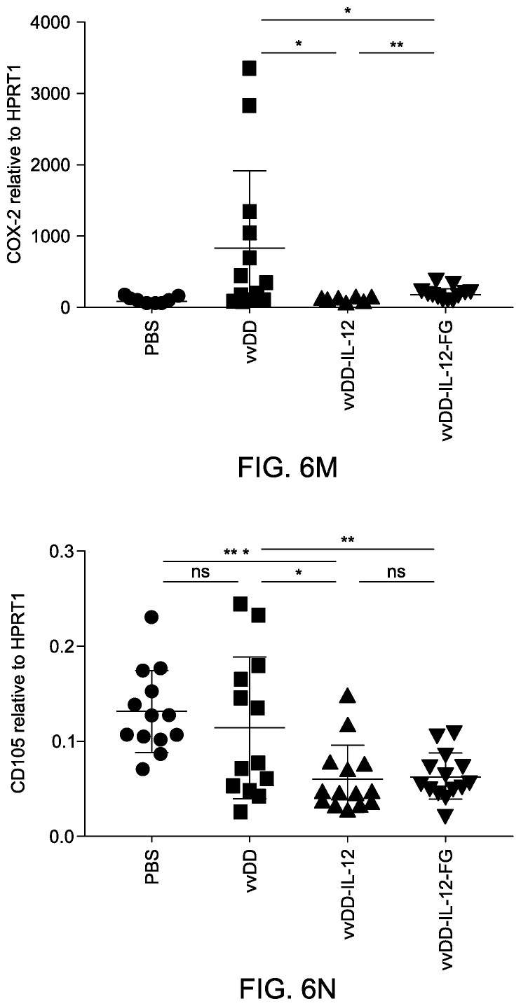

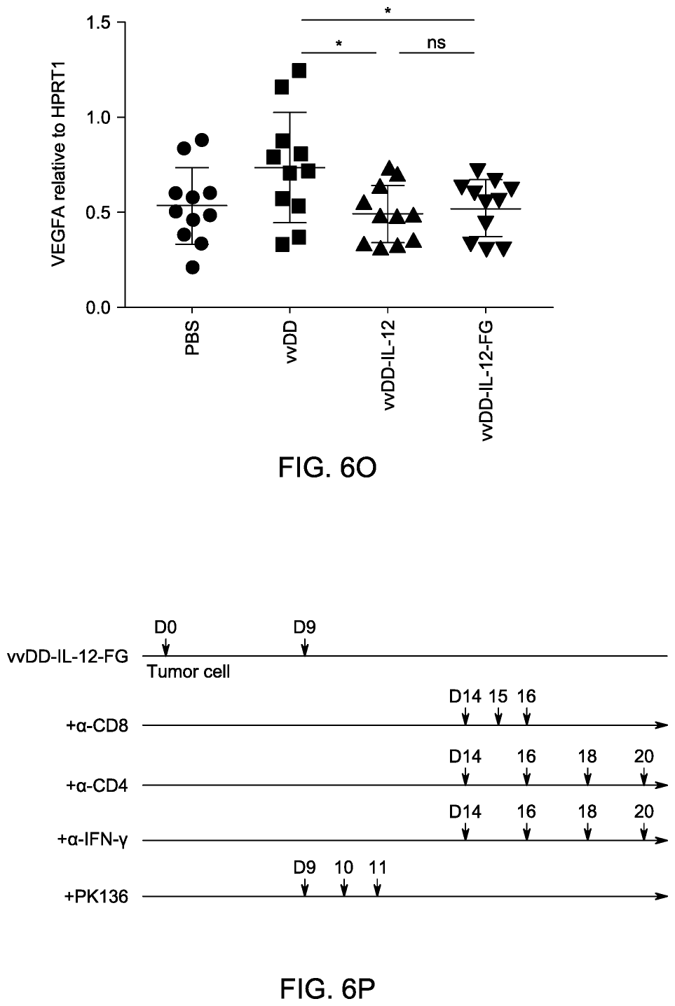

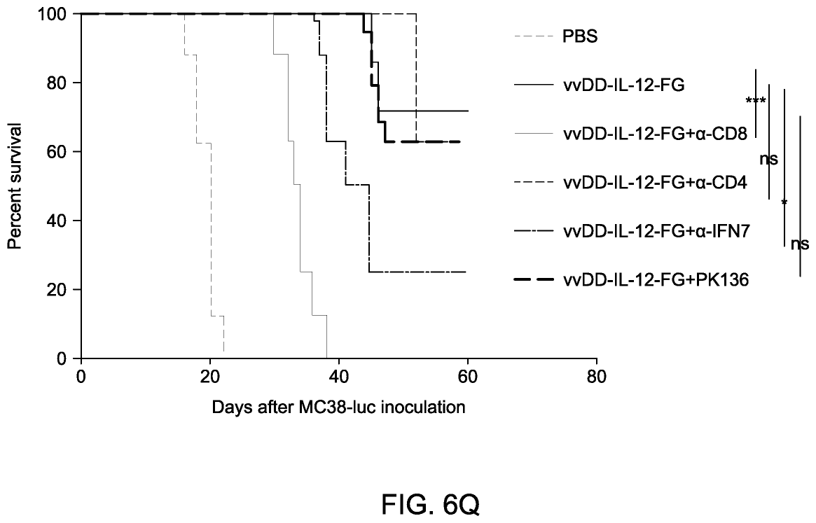

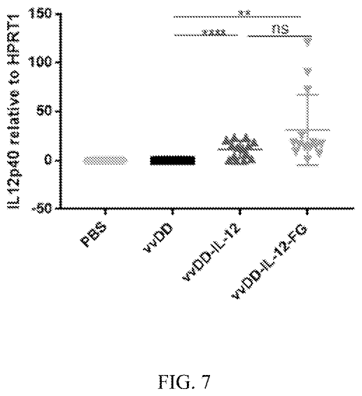

To explore the mechanism by which vvDD-IL-12-FG treatment elicits anti-tumoral immune activity in the profoundly immunosuppressive advanced tumor model, the immune cell profile in the TME was investigated using the late-stage tumor model. The percentages of activated CD4⁺Foxp3⁻and CD8⁺ T cells from tumors receiving IL-12-armed virus treatment were increased compared to those treated with PBS or vvDD (FIGS. 6A and 6B). The results also showed that the more severely exhausted PD1⁺Tim-3⁺CD8⁺, PD1⁺TIGIT⁺CD8⁺, and PD1⁺LAG-3⁺CD8⁺ T cells in the tumor-infiltrating CD8⁺ T cell population decreased after IL-12-armed virus treatment (FIG. 6C-6E). Myeloid-derived suppressor cells (MDSCs) were examined in tumors after virus treatment. It was found that granulocytic MDSCs (G-MDSCs) were increased after vvDD treatment; however, they were decreased after IL-12-armed virus treatment (FIG. 6F). Moreover, the CD8⁺/G-MDSC ratio was significantly higher after IL-12-armed virus treatment compared to other treatments (FIG. 6G). Regulatory T cells (Tregs) were also examined, and it was found that the percentage of CD4⁺Foxp3⁺ T cells in tumor-infiltrating CD4+ T cells were decreased after IL-12-armed virus treatment (FIG. 6H), implying suppression of tumor-induced Treg proliferation by the IL-12/IFN-γ axis, owing to the significantly higher IL-12 (FIG. 7) and IFN-γ levels in tumors that received IL-12-armed virus treatments (FIG. 6I). Correlating with the high IL-12 tethered in the TME (FIG. 4C and FIG. 7), vvDD-IL-12-FG treatment also led to more IFN-γ in the tumor mass (FIG. 6I). However, the increase of IFN-γ in the TME did not upgrade the expression of PD-1 (FIG. 6J) or PD-L1 (FIG. 6K) in tumor compared to those treated with vvDD, suggesting that armed IL-12 did not reinforce adaptive immune resistance in vvDD-related therapy. A significant decrease in the expression of pro-cancer factors, including TGF-β, cyclooxygenase-2 Cox-2, and angiogenesis markers (CD105 and VEGF) was found in tumors after IL-12-armed virus treatment compared with other treatments (FIG. 6L-60). IFN-γ, NK1.1⁺ cells, and CD4⁺ and CD8⁺ T cells were further depleted by antibodies post vvDD-IL-12-FG treatment (FIG. 6P), and it was found that the antitumor effect elicited by vvDD-IL-12-FG treatment was IFN-γ- and CD8⁺ T cell-dependent, but not CD4⁺ T cell- or NK1.1⁺cell-dependent (FIG. 6Q). Collectively, these results demonstrated that vvDD-IL-12-FG treatment, as well as vvDD-IL-12 treatment, tipped the cancer-immune set point in tumor-bearing mice and turned “cold” tumors to “hot” tumors, which significantly extended the survival of mice receiving IL-12-armed virus treatment.

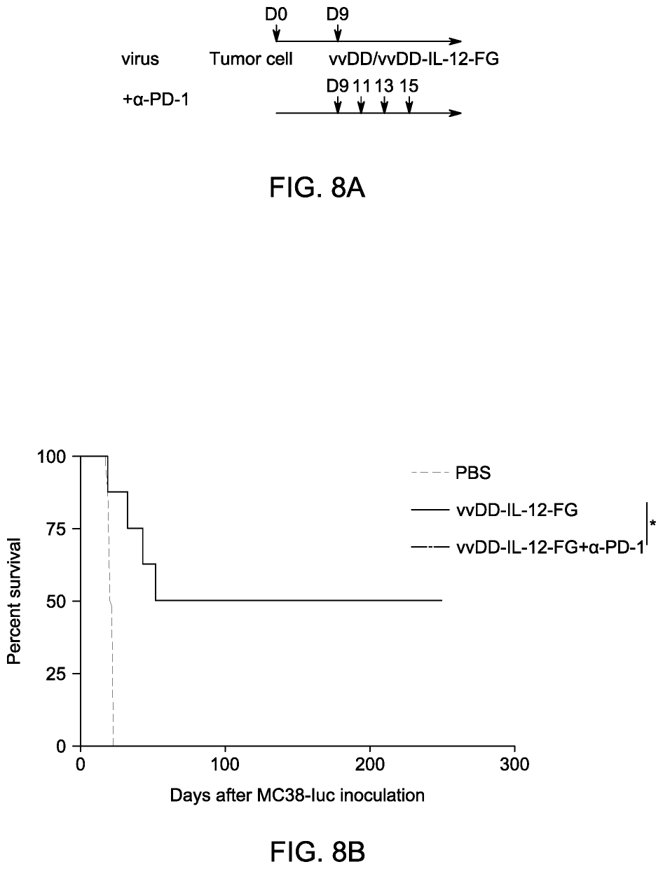

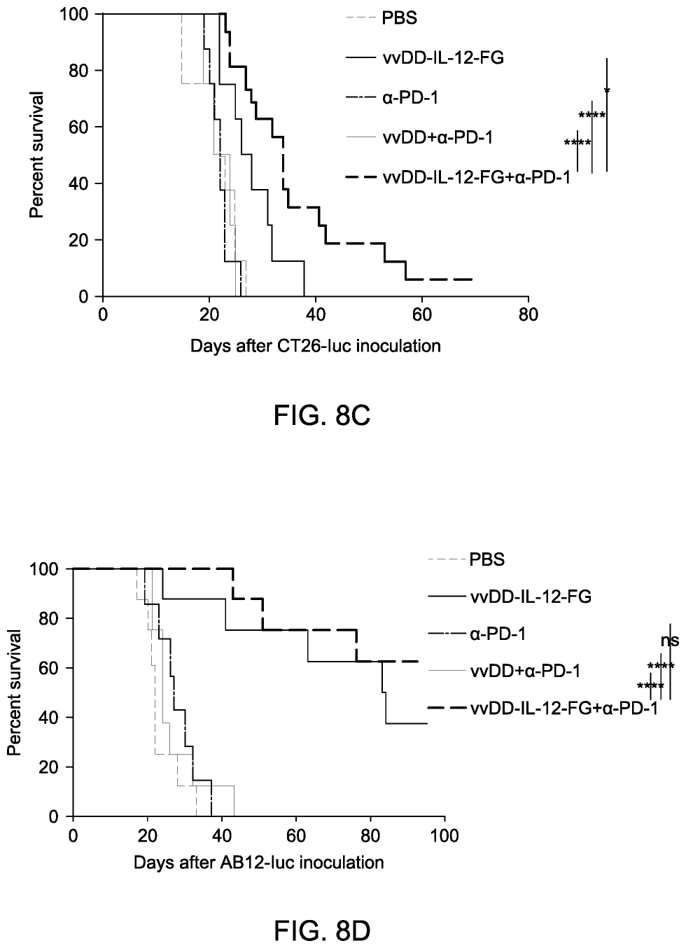

It was tested whether the combination of vvDD-IL-12-FG and anti-PD-1 antibody could improve the therapeutic effects using the late-stage tumor model. The MC38-luc-bearing mice were treated as scheduled (FIG. 8A), and the survival results showed that in the combination of anti-PD-1 antibody treatment plus vvDD-IL-12-FG treatment cured all the advanced tumor-bearing mice (FIG. 8B). The efficacy of the combination treatment was tested using a non-hypermutated/non-microsatellite-instable colon cancer CT26 model (nine-day-tumor-bearing), and it was found that the combination of vvDD-IL-12-FG and anti-PD-1 antibody treatment also was effective in this less-immunogenic tumor model (FIG. 8C). The efficacy of this combination treatment was further tested using a mesothelioma AB12-luc model (nine-day-tumor-bearing), and the combination treatment improved the therapeutic effect, though not significantly, compared with virus monotherapy (FIG. 8D). These results demonstrated safe, local delivery of tethered IL-12 via an oncolytic virus, combined with immune checkpoint blockade, as an effective cancer immunotherapy agent.

In summary, the results provided herein demonstrated that vvDD-IL-12-FG treatment can deliver IL-12 to the tumor bed and tether IL-12 on cell membranes, which was shown to be safe and effective at modifying the cancer-immune set point and producing an immune-favorable microenvironment, and further improving the efficacy as a monotherapy. Moreover, the combination of vvDD-IL-12-FG and anti-PD-1 antibody treatment induced effective therapeutic effects in various tumor models. In profoundly immunosuppressive, advanced stage disease, vvDD-IL-12-FG synergizes with anti-PD-1 antibody therapy, leading to the cure of all late stage MC38 tumors.

Taken together, these results demonstrate that vvDD-IL-12-FG can be used as a new form of IL-12 immunotherapy, representing a treatment for cancers that have historically been unresponsive to immune checkpoint blockade-based immunotherapy.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A recombinant vaccinia virus comprising a vaccinia virus genome comprising (a) nucleic acid encoding a first polypeptide and (b) nucleic acid encoding a second polypeptide, wherein said first polypeptide comprises an IL-12p35 polypeptide sequence, wherein said second polypeptide comprises an IL-12p40 polypeptide, and wherein said first polypeptide or said second polypeptide comprises a membrane anchoring polypeptide sequence.

2. The recombinant vaccinia virus of claim 1, wherein said IL-12p35 polypeptide sequence is a full length human IL-12p35 polypeptide sequence or a full length mouse IL-12p35 polypeptide sequence.

3. (canceled)

4. The recombinant vaccinia virus of claim 1, wherein said IL-12p40 polypeptide sequence is a full length human IL-12p40 polypeptide sequence or a full length mouse IL-12p40 polypeptide sequence.

5. (canceled)

6. The recombinant vaccinia virus of claim 1, wherein said membrane anchoring polypeptide sequence comprises a polypeptide having a glycosylphosphatidyl-inositol (GPI) modification.

7. The recombinant vaccinia virus of claim 6, wherein said membrane anchoring polypeptide sequence is from about 10 amino acids to about 50 amino acids in length.

8. The recombinant vaccinia virus of claim 6, wherein said polypeptide having a GPI modification is derived from a CD16b polypeptide.

9. (canceled)

10. The recombinant vaccinia virus of claim 1, wherein said first polypeptide comprises said membrane anchoring polypeptide sequence, and wherein said first polypeptide comprises a polypeptide linker between said IL-12p35 polypeptide sequence and said membrane anchoring polypeptide sequence or wherein said second polypeptide comprises said membrane anchoring polypeptide sequence wherein said second polypeptide comprises said polypeptide linker between said IL-12p40 polypeptide sequence and said membrane anchoring polypeptide sequence.

11-13. (canceled)

14. The recombinant vaccinia virus of claim 10, wherein said polypeptide linker is from about one amino acid to about 25 amino acids in length.

15. The recombinant vaccinia virus of claim 14, wherein said polypeptide linker comprises a (G₄S)₃ sequence or an A(EA₃K)₄AAA (SEQ ID NO:14) sequence.

16. (canceled)

17. The recombinant vaccinia virus of claim 1, wherein said nucleic acid encoding said first polypeptide is operably linked to a promoter capable of driving transcription of a polycistronic transcript that expresses said first polypeptide and said second polypeptide.

18. The vaccinia virus of claim 17, wherein said promoter is selected from the group consisting of a p7.5 e/1 promoter and a pSe/1 promoter.

19. The recombinant vaccinia virus of claim 17, wherein said nucleic acid encoding said first polypeptide and said nucleic acid encoding said second polypeptide are separated by an internal ribosome entry site (IRES).

20. The recombinant vaccinia virus of claim 1, wherein a cell expressing said first polypeptide and said second polypeptide expresses said first polypeptide and said second polypeptide on its surface in the form of a heterodimer having the ability to stimulate an IL-12 receptor of another cell.

21. A method for treating a mammal having cancer, wherein said method comprises administering, to said mammal, a recombinant vaccinia virus of claim 1, wherein said recombinant vaccinia virus is capable of infecting a cell and expressing a membrane-bound IL-12 polypeptide comprising said first polypeptide and said second polypeptide on a surface of said cell.

22. The method of claim 21, wherein said mammal is a human.

23. The method of claim 21, wherein said cell is a cancer cell or a stromal cell in a tumor microenvironment of said mammal.

24. (canceled)

25. The method of claim 21, wherein said cancer is selected from the group consisting of colon cancer, lung cancer, prostate cancer, ovarian cancer, hepatocellular carcinoma, pancreatic cancer, kidney cancer, melanoma, brain cancer, lymphoma, myeloma, lymphocytic leukemia, myelogenous leukemia, and breast cancer.

26-29. (canceled)

30. A method for increasing the number of activated T cells within a tumor microenvironment present in a mammal, wherein said method comprises administering, to said mammal, a recombinant vaccinia virus of claim 1, wherein a cell within said mammal expresses a membrane-bound IL-12 polypeptide comprising said first polypeptide and said second polypeptide on its surface, and wherein the number of activated T cells within said tumor microenvironment is increased.

31. The method of claim 30, wherein said mammal is a human.

32. The method of claim 30, wherein said activated T cell is selected from the group consisting of CD4+ T cells, CD8+ T cells, and natural killer T cells.

33. A method for decreasing the number of suppressor T cells within a tumor microenvironment present in a mammal, wherein said method comprises administering, to said mammal, a recombinant vaccinia virus of claim 1, wherein a cell within said mammal expresses a membrane-bound IL-12 polypeptide comprising said first polypeptide and said second polypeptide on its surface, and wherein the number of suppressor T cells within said tumor microenvironment is decreased.

34. The method of claim 33, wherein said mammal is a human.

35. The method of claim 33, wherein said suppressor T cell is selected from the group consisting of regulatory T cells (Tregs), granulocytic myeloid-derived suppressor cells (G-MDSCs), and exhausted CD8⁺ T cells.
